(19) United States
(12) Patent Application Publication
(10) Pub. No.: US 2012/0270239 A1
(43) Pub. Date: Oct. 25, 2012

(54) MELANOMA PROGNOSTIC MODEL USING TISSUE MICROARRAYS AND GENETIC ALGORITHMS

(76) Inventors: David L. Rimm, Branford, CT (US); Aaron J. Berger, Mountain View, CA (US); Bonnie Rothberg, Guilford, CT (US); Robert L. Camp, San Francisco, CA (US); Harriet Kluger, Woodbridge, CT (US)

(21) Appl. No.: 13/505,240

(22) PCT Filed: Oct. 29, 2010
(86) PCT No.: PCT/US10/54799

§ 371 Date: Jul. 11, 2012

(30) Foreign Application Priority Data
Oct. 30, 2009 (US) 61256339

(51) Int. Cl. G01N 33/574; G01N 21/64

(52) U.S. Cl. 435/7.23

(57) ABSTRACT

The invention provides a method for determining the risk that a patient diagnosed with melanoma will develop a recurrence of melanoma comprising: a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient; and b) determining whether an expression parameter for each marker in the tumor tissue sample is achieved by comparing the level of expression of each marker with a predetermined reference level associated with each marker; wherein the patient is at a low risk of developing a recurrence of melanoma if four or more of the expression parameters are achieved and wherein the patient is at a high risk of developing a recurrence of melanoma if three or fewer of the expression parameters are achieved.

This application claims priority of U.S. Provisional Application No. 61/256,339, filed Oct. 30, 2009, the entire content of which is hereby incorporated by reference into this application.

This invention was made with support under R01 CA114277 and P50 CA121974 awarded by the National Institute of Health. Accordingly, the United States government has certain rights in the invention.

Throughout this application, various publications are referenced by endnotes and/or Arabic numerals within parentheses. Full citations for these publications may be found at the end of the specification immediately preceding the claims. The disclosures of each of these publications is hereby incorporated by reference into this application in order to more fully describe the state of the art as known to those skilled therein as of the date of this application.

### FIELD OF THE INVENTION

This invention relates to the field of a melanoma prognostic model using tissue microarrays and genetic algorithms.

### BACKGROUND OF THE INVENTION

Adjuvant therapy is the standard of care for many low stage cancers that can be completely resected with tumor-free margins. However, for some other cancers, the lack of effective and safe adjuvant therapy leads to an excess of mortality directly related to the development of metastatic disease in patients assumed to have undergone a complete resection of their malignancy. One important example is cutaneous malignant melanoma, the 6^{th} most common cancer in the US¹. Although over 80% of new cases are still localized to the skin¹ where a wide local excision should be curative in the setting of a negative sentinel lymph node biopsy, the unfavorable risk-benefit ratio of available adjuvant regimens advocates caution when administering such agents to individuals with Stage I-IIA and even in Stage IIB or IIC, where high-dose interferon-alfa-2b is currently US Food and Drug Administration-approved in the adjuvant setting². Consequently, 20% of these patients will develop metastases and die of their disease within 10 years with over 30% 10-year mortality among those with T3 and T4 tumors³. Development of a prognostic tool that could selectively triage the subset of high recurrence risk Stage II patients for adjuvant therapy could potentially lower the burden of untreatable metastatic cancer, and enable us to selectively treat those patients that are more likely to develop distant metastatic disease.

Nine clinicopathologic prognostic markers have been identified and incorporated in clinically validated outcome risk stratification models^{3,4}. However, these do not account for all of the observed variability associated with melanoma-related survival. Immunohistochemistry (IHC) is a widely-accepted and well-documented method for characterizing patterns of protein expression in formalin-fixed, paraffin-embedded (FFPE) samples while preserving tissue and cellular architecture⁵. Although no IHC marker has become standard of care, new work may suggest the inclusion of Ki-67⁶. Our recent systematic review of melanoma IHC data shows that individual contributions of IHC markers to overall prognosis are of narrow statistical significance and thus unlikely to demonstrate broad clinical utility⁷ or see wide adoption.

Here, we describe the generation of an independently significant, multi-marker prognostic model for melanoma using genetic algorithms on a subset of 38 candidate proteins assessed upon a cohort of 192 primary melanomas. Our model shows 2 prognostic groups (low risk and high risk), created from 5 markers, that successfully validated as a significant independent prognostic factor in a second cohort of 246 primary melanomas. These data demonstrate the potential for multi-marker assays in improving melanoma prognostic assessment and warrants a prospective, randomized, controlled melanoma prognostic study. This test could be a valuable tool to help determine which sentinel node-negative stage II melanoma patients should seek adjuvant therapy or other aggressive management strategies.

### SUMMARY OF THE INVENTION

The invention provides a method for determining the risk that a patient diagnosed with melanoma will develop a recurrence of melanoma comprising: a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient; and b) determining whether an expression parameter for each marker in the tumor tissue sample is achieved by comparing the level of expression of each marker with a predetermined reference level associated with each marker; wherein the patient is at a low risk of developing a recurrence of melanoma if four or more of the expression parameters are achieved and wherein the patient is at a high risk of developing a recurrence of melanoma if three or fewer of the expression parameters are achieved.

The invention provides a method for determining the risk that a patient diagnosed with melanoma will develop metastatic disease comprising: a) determining the level of expression for each marker of a panel of markers, wherein the panel comprises activating transcription factor 2, p21^{WAF1}, p16^{INK4A}, ⊕-catenin, and fibronectin and the levels of expression are determined in compartments of interest in cells of interest in a tumor tissue sample from the patient; and b)determining whether an expression parameter for each marker in the tumor tissue sample is achieved by comparing the level of expression of each marker with a predetermined reference level associated with each marker; wherein the patient is at a low risk of developing metastatic disease if four or more of the expression parameters are achieved and wherein the patient is at a high risk of developing metastatic disease if three or fewer of the expression parameters are achieved.

The invention provides a method for determining the risk that a patient diagnosed with melanoma will develop a recurrence of melanoma which comprises: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing a recurrence of melanoma if four or more of the parameters are achieved and wherein the patient is at a high risk of developing a recurrence of melanoma if three or fewer of the parameters are achieved.

The invention provides a method for determining the risk that a patient diagnosed with melanoma will develop metastatic disease which comprises: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{INK4A} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing metastatic disease if four or more of the parameters are achieved and wherein the patient is at a high risk of developing metastatic disease if three or fewer of the parameters are achieved.

The invention provides a method for classifying a patient diagnosed with melanoma as being low risk for a recurrence of melanoma comprising: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing a recurrence of melanoma if four or more of the parameters are achieved.

The invention provides a method for classifying a patient diagnosed with melanoma as being high risk for a recurrence of melanoma comprising: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a high risk of developing a recurrence of melanoma if three or fewer of the parameters are achieved.

The invention provides a method for determining whether a patient diagnosed with melanoma is likely to benefit from adjuvant therapy comprising: a) determining the level of expression of activating transcription factor 2 present within the nuclear compartment and the non-nuclear compartment in cells of interest in a tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tissue sample; d) determining the level of expression of p16^{INK4A} present within the nuclear compartment and the non-nuclear compartment in the cells of interest in the tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tissue sample; g) determining the level of expression of fibronectin present within the nuclear and non-nuclear compartments combined in the cells of interest in the tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is likely to benefit from adjuvant therapy if three or fewer of the parameters are achieved.

The invention provides a kit comprising a first stain specific for activating transcription factor 2; a second stain specific for p21^{WAF1}; a third stain specific for p16^{INK4A}; a fourth stain specific for β-catenin; a fifth stain specific for fibronectin; a sixth stain specific for a subcellular compartment of a cell; and instructions for using the kit.

### BRIEF DESCRIPTION OF THE FIGURES

FIG. 1: Kaplan-Meier estimates of melanoma-specific mortality among the 129 Yale Melanoma Discovery Cohort participants with complete data across the 5 markers comprising the genetic-algorithm-based multi-marker prognostic assay according to algorithm-derived prognostic score. A. Survival curves drawn according to number of prognostic conditions met. B. Survival curves for the dichotomized model describing low-risk (4-5 conditions met) or high-risk (≦3 conditions met) groupings.

FIG. 2: Kaplan-Meier estimates of melanoma-specific mortality for the dichotomized model describing favorable or unfavorable profiles among: A. all 226 participants of the Yale Melanoma Validation Cohort scored completely for the multi-marker prognostic assay and B. the 193 members of the Yale Melanoma Validation Cohort who are sentinel lymph node negative (Stage II melanoma).

### DETAILED DESCRIPTION OF THE INVENTION

A “predetermined reference level” associated with a particular biomarker and a “predetermined reference ratio” associated with a particular biomarker refers to a cut-point associated with a particular biomarker.

A “reference ratio” may refer to a ratio of the level of expression of a particular biomarker within a non-nuclear compartment relative to the level of expression of a particular biomarker within a nuclear compartment wherein the former is the numerator and the latter is the denominator.

The invention provides a method for determining the risk that a patient diagnosed with melanoma will develop a recurrence of melanoma comprising: a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient; and b) determining whether an expression parameter for each marker in the tumor tissue sample is achieved by comparing the level of expression of each marker with a predetermined reference level associated with each marker; wherein the patient is at a low risk of developing a recurrence of melanoma if four or more of the expression parameters are achieved and wherein the patient is at a high risk of developing a recurrence of melanoma if three or fewer of the expression parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p21^{WAF1}, p16^{INK4A}, β-catenin, and fibrectin may be determined using a quantitative image analysis procedure.

Numerous quantitative image analysis procedures are known in the art.

An example of a quantitative image analysis procedures that may be used to determine the level of expression include AQUA® analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The compartments of interest may be the nuclear compartment and the non-nuclear compartment.

The invention provides a method for determining the risk that a patient diagnosed with melanoma will develop metastatic disease comprising: a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient; and b)determining whether an expression parameter for each marker in the tumor tissue sample is achieved by comparing the level of expression of each marker with a predetermined reference level associated with each marker; wherein the patient is at a low risk of developing metastatic disease if four or more of the expression parameters are achieved and wherein the patient is at a high risk of developing metastatic disease if three or fewer of the expression parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p16^{INK4A}, β-catenin, and fibrectin may be determined using a quantitative image analysis procedure.

Numerous quantitative image analysis procedures are known in the art. An example of a quantitative image analysis procedures that may be used to determine the level of expression include AQUA® analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The compartments of interest may be the nuclear compartment and the non-nuclear compartment.

The invention provides a method for determining the risk that a patient diagnosed with melanoma will develop a recurrence of melanoma which comprises: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing a recurrence of melanoma if four or more of the parameters are achieved and wherein the patient is at a high risk of developing a recurrence of melanoma if three or fewer of the parameters are achieved.

The levels of expression of activating transcription factor 2, p21^{WAF1}, β-catenin, and fibrectin may be determined using an automated pathology system.

The levels of expression of activating transcription factor 2, p21^{WAF1}, p16^{INK4A}, β-catenin, and fibrectin may be determined using quantitative image analysis procedure.

Numerous quantitative image analysis procedures are known in the art. An example of a quantitative image analysis procedures that may be used to determine the level of expression include AQUA® analysis, as described in issued U.S. Pat. No. 7,219,016, and in U.S Patent

Application Publication No. 2009/0034823, which are incorporated by reference into this application in its entirety.

The melanoma may be a stage II cancer.

The patient diagnosed with melanoma may be lymph node negative.

The invention provides a method for determining the risk that a patient diagnosed with melanoma will develop metastatic disease which comprises: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A}present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing metastatic disease if four or more of the parameters are achieved and wherein the patient is at a high risk of developing metastatic disease if three or fewer of the parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p21^{WAF1}, p16^{INK4A}, β-catenin, and fibrectin may be determined using a quantitative image analysis procedure.

Numerous quantitative image analysis procedures are known in the art. An example of a quantitative image analysis procedures that may be used to determine the level of expression include AQUA® analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The invention provides a method for classifying a patient diagnosed with melanoma as being low risk for a recurrence of melanoma comprising: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a low risk of developing a recurrence of melanoma if four or more of the parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p21^{WAF1}, p16^{INK4A}, β-catenin, and fibrectin may be determined using a quantitative image analysis procedure.

Numerous quantitative image analysis procedures are known in the art. An example of a quantitative image analysis procedures that may be used to determine the level of expression include AQUA analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The invention provides a method for classifying a patient diagnosed with melanoma as being high risk for a recurrence of melanoma comprising: a) determining the level of expression of activating transcription factor 2 present within a nuclear compartment and a non-nuclear compartment in cells of interest in a tumor tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tumor tissue sample; d) determining the level of expression of p16^{INK4A} present within the nuclear compartment and the non-nuclear compartment in the cells of interest in the tumor tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tumor tissue sample; g) determining the level of expression of fibronectin present within the nuclear and non-nuclear compartments combined in the cells of interest in the tumor tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is at a high risk of developing a recurrence of melanoma if three or fewer of the parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p21^{WAF1}, p16^{INK4A}, β-catenin, and fibrectin may be determined using quantitative image analysis procedure.

Numerous quantitative image analysis procedures are known in the art. An example of a quantitative image analysis procedures that may be used to determine the level of expression include AQUA® analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The invention provides a method for determining whether a patient diagnosed with melanoma is likely to benefit from adjuvant therapy comprising: a) determining the level of expression of activating transcription factor 2 present within the nuclear compartment and the non-nuclear compartment in cells of interest in a tissue sample from the patient; b) obtaining a ratio of the level of expression of activating transcription factor 2 present within the non-nuclear compartment relative to the level of expression of activating transcription factor 2 present within the nuclear compartment; c) determining the level of expression of p21^{WAF1} present within the nuclear compartment in the cells of interest in the tissue sample; d) determining the level of expression of p16^{INK4A} present within the nuclear compartment and the non-nuclear compartment in the cells of interest in the tissue sample; e) obtaining a ratio of the level of expression of p16^{INK4A} present within the non-nuclear compartment relative to the level of expression of p16^{INK4A} present within the nuclear compartment; f) determining the level of expression of β-catenin present within the nuclear and non-nuclear compartments combined in the cells of interest in the tissue sample; g) determining the level of expression of fibronectin present within the nuclear and non-nuclear compartments combined in the cells of interest in the tissue sample; h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2; i) comparing the level of expression obtained in step c) to a predetermined reference level associated with p21 wherein the parameter for p21^{WAF1} is achieved if the level of expression obtained in step c) is greater than the predetermined reference level of expression associated with p21^{WAF1}; j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A}; k) comparing the level of expression obtained in step f) to a predetermined reference level associated with β-catenin wherein the parameter for β-catenin is achieved if the level of expression obtained in step f) is greater than the predetermined reference level of expression associated with β-catenin; and l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin; wherein the patient is likely to benefit from adjuvant therapy if three or fewer of the parameters are achieved.

The levels of expression of activating transcription factor 2, p21^{WAF1}, p16^{INK4A}, β-catenin, and fibrectin may be determined using an automated pathology system.

The levels of expression of activating transcription factor 2, p21^{WAF1}, p16^{INK4A}, β-catenin, and fibrectin may be determined using a quantitative image analysis procedure.

Numerous quantitative image analysis procedures are known in the art. An example of a quantitative image analysis procedures that may be used to determine the level of expression include AQUA® analysis, as described in issued U.S. Pat. No. 7,219,016, and in U.S Patent Application Publication No. 2009/0034823, which are incorporated by reference into this application in its entirety.

The melanoma may be a stage II cancer.

The patient diagnosed with melanoma may be lymph node negative.

The invention provides a kit comprising a first stain specific for activating transcription factor 2; a second stain specific for p21^{WAF1}; a third stain specific for p16^{INK4A}; a fourth stain specific for β-catenin; a fifth stain specific for fibronectin; a sixth stain specific for a subcellular compartment of a cell; and instructions for using the kit.

The kit may be further comprised of predetermined reference level values associated with each of activating transcription factor 2, p21^{WAF1}, p16^{INK4A}, β-catenin, and fibronectin

Experimental Details

Part 1

### SUMMARY OF THE INVENTION

Described is a method to estimate the probability that a patient diagnosed with melanoma will develop a recurrence of this disease. This information is useful to the patient and the physician for assessing the risk versus benefit of observation versus adjuvant therapy for a particular patient.

The method as described involves the quantitative immunofluorescence (QIF) signal of five protein markers in a sample of the patient's primary invasive cutaneous melanoma. Accurate quantification of the QIF signal for each of the five markers was achieved using the automated quantitative analysis (AQUA) technology as previously described. The AQUA technology permits quantification not only of the fluorescence signal for a given marker within the tissue sample under analysis, but also permits accurate compartmentalization within molecularly defined subcellular compartments, which are critical for this test. The subject invention of this disclosure relates to 1) the markers that have been identified, 2) the algorithm applied to the relative levels of each of the five markers, 3) the cut-off levels of the five markers, and 4) the method of standardization to reproducibly define these cut-off values. While the AQUA platform is likely the easiest method to generate the components of this assay, it is envisioned that this invention is also applicable to alternative platform technologies capable of quantifying the markers as described.

The five markers are: activating transcription factor 2 (ATF2), p21^{WAF1}, p16^{INK4A}, β-catenin, and fibronectin. The levels of expression of these markers are determined in cellular components of interest as follows:
(i) ATF2 expression levels are measured in both the non-nuclear and nuclear compartments and a ratio of non-nuclear:nuclear expression is determined;
(ii) p21^{WAF1} expression level in measured in the nuclear compartment
(iii) p16^{INK4A} expression levels are measured in the non-nuclear and nuclear compartments and the ratio of non-nuclear:nuclear expression is determined;
(iv) β-catenin expression levels are measured in both the non-nuclear and nuclear compartments combined
(v) fibronectin expression levels are measured in both the non-nuclear and nuclear compartments combined.

In one embodiment of the invention, specifically as quantified using the AQUA technology, the cut-off levels for each of these parameters is as follows:
(i) ATF2 ratio greater than −0.052
(ii) p21^{WAF1} nuclear compartment levels greater than 12.98
(iii)p16^{INK4A} ratio less than or equal to −0.083
(iv) total β-catenin level greater than 38.68
(v) total fibronectin level less than or equal to 57.93

Based on these markers and these cut-off levels using the AQUA technology, a patient is classified as being low risk for earlier recurrence of the melanoma if analysis demonstrated that the cut-off level was achieved for at least four of these five parameters in the patient's primary tumor. Conversely, a patient whose primary tumor demonstrates three or fewer of these parameters is classified as being high risk for early recurrence of the melanoma.

Analysis of the levels of these five markers using alternative platform technologies is also envisioned, although standards and standardization methods disclosed herein would be required for accurate translation of this test to whole sections and to platforms other than the AQUA platform. When alternative technologies are used, optimized cutpoint values for the five markers can be derived utilizing the alternative platforms using the methods described herein for deriving cutpoints.

The clinical application of this invention would provide an objective assessment of a patient's likelihood of early recurrence of the disease that is complementary to existing criteria. Based on this information, patients most likely to benefit from adjuvant therapy or from a more aggressive monitoring of disease recurrence, can be identified. Conversely, patients who may be candidates for adjuvant therapy based on current prognostic criteria, but who are identified as being at low risk for recurrence based on this assay, may avoid the unnecessary risks associated with existing adjuvant therapy.

Part 2

Abstract

Purpose: Due to the questionable risk/benefit ratio of adjuvant therapies, Stage II melanoma is currently managed by observation as available clinicopathologic parameters cannot identify the 20-60% of such patients likely to develop metastatic disease. Here, we propose a multi-marker molecular prognostic assay that can help triage patients at increased risk of recurrence.

Methods: Protein expression for 38 candidates relevant to melanoma oncogenesis was evaluated using the AQUA method for immunofluorescence-based immunohistochemistry in formalin fixed, paraffin embedded specimens from a cohort of 192 primary melanomas collected during 1959-1994. The prognostic assay was built using a genetic algorithm and validated on an independent cohort of 246 serial primary melanomas collected from 1997-2004.

Results: Multiple iterations of the genetic algorithm yielded a consistent 5-marker solution. A favorable prognosis was predicted by: ATF2 ln(non-nuclear/nuclear AQUA score ratio) >−0.052, p21^{WAF1} nuclear compartment AQUA score>12.98, p16^{INK4A} ln(non-nuclear/nuclear AQUA score ratio) ≦−0.083, β-catenin total AQUA score>38.68, and fibronectin total AQUA score≦57.93. Primary tumors that met at least 4 of the 5 conditions above were considered a “low risk” group and those that met 3 or fewer conditions formed a “high risk” group (log rank p<0.0001). Multivariable proportional hazards analysis adjusting for clinicopathologic parameters shows that the high-risk group has significantly reduced survival on both the Discovery (HR=2.84; 95% CI=1.46-5.49; p=0.002) and Validation (HR=2.72, 95% CI=1.12-6.58; p=0.027) cohorts.

Conclusions: This multi-marker prognostic assay, an independent determinant of melanoma survival, might be beneficial in improving the selection of stage II patients for adjuvant therapy.

Methods

Patients and Tumor Samples

Seven-hundred and thirty-seven tumor samples from three non-overlapping series of patients with cutaneous melanoma were analyzed for protein expression. The Yale Melanoma Discovery Cohort consisted of 192 Caucasian patients who underwent resection of a primary invasive cutaneous melanoma at Yale-New Haven Hospital during 1959-1994 for whom the surgical specimen was not exhausted during diagnosis and for which follow-up information is available. The Yale Melanoma Validation Cohort included 246 serial Clark levels III-V cutaneous melanoma patients who underwent sentinel lymph node biopsy by a single surgeon during 1997-2004⁸. The Yale Metastatic Series includes 299 unique subcutaneous metastases, lymph node metastases or visceral metastases occurring in patients previously diagnosed with cutaneous melanoma and surgically removed at Yale-New Haven Hospital during 1959-1994 (n=198) or during 1995-2002 (n=101). For the primary melanomas, clinical data describing patient demographics, date of diagnosis, clinical course and follow-up through Aug. 1, 2007 were obtained following a comprehensive review of the medical record, the archives of the Connecticut Tumor Registry and, if applicable, the State of Connecticut Vital Records. Stage at diagnosis (localized, regional and distant) and anatomic location were obtained from the surgical report. Receipt of non-surgical therapy referred to administration of cytotoxic chemotherapy, immunomodulators or radiotherapy either in the adjuvant setting or following clinical recurrence. For each cohort a single investigator reviewed all slides to reconfirm the diagnosis of melanoma and to determine Breslow thickness, Clark level of invasion, histopathologic subtype, and the presence of ulceration, microsatellitosis and tumor-infiltrating lymphocytes. This study was approved by the Yale Human Investigations Committee.

Tissue Microarray Construction, Immunohistochemistry and Automated Image Acquisition and Analysis (AQUA®)

Formalin-fixed, paraffin-embedded (FFPE) blocks were retrieved from the Yale Pathology Archives and 0.6 mm tissue microarrays (TMAs) were constructed according to the published method⁹. The discovery TMA included single cores from the 192 primary melanomas, the 299 metastases along with a series of controls. The validation TMA included two-fold redundant cores in separate blocks from the 246 cases plus a random selection of 60 individuals from the discovery series to facilitate normalization of the validation array. Fluorescence-based immunohistochemical staining was performed by standard procedures¹⁰(See Supplemental Methods).

AQUA image and acquisition analysis was performed as previously described¹¹. Briefly, stained histospots were imaged and regions of tumor were defined by an S100B binary signal. Within the tumor region, the nuclear compartment is identified as the subset of pixels that demonstrated any DAPI staining within the plane of focus. The non-nuclear compartment is then indicated as all pixels assigned to the tumor mask but not included within the nuclear compartment. Finally, the target antigen expression is automatically determined, blinded to any a priori clinical information, as the sum of intensities from the Cy5 channel in all pixels within a compartment divided by the number of pixels.

Statistical Analysis

Cores whose tumor mask covered <5% of the total histospot area were dropped from further analysis. For individuals represented by multiple cores on the TMA, AQUA scores were averaged prior to analysis. To normalize the AQUA scores between the discovery and validation cohorts, a regression equation was calculated for the set of 60 samples spotted on both arrays and the mean values for the validation cohort were adjusted according to the regression equation.

To develop a multi-marker prognostic model from the discovery cohort data, a genetic algorithm using standard methodology^{12,13} within the X-tile software suite¹⁴ (see Supplemental Methods) with a 33% crossover and 33% mutation rate constrained to create a multi-marker profile that included a minimum of 100/192 eligible individuals with complete data across all selected markers was created. Additional algorithm specifications limited individual marker cut-points to include ≧10% of the available population in each arm and required that each category defined by the marker groupings both contain no fewer than 15% of the available population and, to maintain statistical robustness of the final model, enumerate no fewer than 2 events of interest. We did not constrain the number of parameters to be included in the selected model. Briefly, the algorithm randomly selects a set of markers and, for each marker, chooses a random cut-point to binarize the continuous AQUA data, where, by convention, a score of 1 indicates reduced risk and 0 indicates increased risk. Next, for each individual, the binary marker scores are summed and the log-rank statistic for melanoma-specific survival is calculated across all marker sum categories. This initial seed model is then subjected to multiple iterations by either “mutation” (altering the cut-point for an already-included marker) or by “cross-over” (swapping among the set of eligible markers) until the model converges on a set of markers and their respective cut-points that yield the highest log-rank Chi-square statistic for melanoma-specific survival, typically achieved between 16 and 18 million iterations.

Five parallel iterations of the genetic algorithm were executed. Melanoma-specific survival was the end-point for all survival analyses; individuals who died from competing causes were censored at the time of death. All underlying assumptions for regression and survival analyses were verified using stand procedures. Bivariate and survival analyses were performed using SAS version 9.1.3 and Statview 5.0 (SAS Institute, Cary, N.C.) and adjustments for multiple comparisons executed by the standard Bonferroni method.

Results

Patient Characteristics

The distribution of demographic and clinicopathologic characteristics for both the Discovery and Validation cohorts is presented (Table 1). In addition to the longer follow-up time (p<0.0001), the Discovery cohort displayed overall thicker tumors (p=0.01), a more balanced gender distribution (p=0.04), a higher prevalence of ulcerated melanomas (p=0.01), and fewer superficial spreading melanomas (p=0.04) than the Validation cohort.

TABLE 1
Characteristics of the Yale Melanoma Discovery and Validation cohorts

| | Discovery | Validation | |
|---|---|---|---|
| | Cohort | Cohort | |
| Parameter | (n = 192)* | (n = 246) | p-value |
| Mean follow-up time for | 9.50 ± 9.14 | 4.05 ± 2.12 | p < 0.0001^{†} |
| censored individuals | | | |
| (yrs) | | | |
| Breslow thickness (mm) | 2.42 ± 2.01 | 1.95 ± 1.78 | p = 0.01^{†} |
| Age at diagnosis (yrs) | 57.77 ± 15.65 | 59.28 ± 16.76 | p = 0.34 |
| Gender | | | |
| Male | 96 (50.0%) | 147 (59.8%) | p = 0.04^{†} |
| Female | 96 (50.0%) | 99 (40.2%) | |
| Stage at diagnosis | | | |
| Localized | 160 (84.2%) | 246 (100%) | N/A |
| Regional spread | 16 (8.4%) | — | |
| Distant metastases | 14 (7.4%) | — | |
| Ulceration | | | |
| Absent | 135 (70.3%) | 198 (80.5%) | p = 0.01^{†} |
| Present | 57 (29.7%) | 48 (19.5%) | |
| Tumor-infiltrating | | | |
| lymphocytes | | | |
| Non-brisk | 150 (78.5%) | 208 (84.9%) | p = 0.09 |
| Brisk | 41 (21.5%) | 37 (15.1%) | |
| Histologic subtype | | | |
| Superficial spreading | 127 (66.1%) | 132 (73.7%) | p = 0.04^{†} |
| Nodular | 30 (15.6%) | 24 (13.4%) | |
| Lentigo maligna | 8 (4.2%) | 4 (2.2%) | |
| Acral lentiginous | 11 (5.7%) | 1 (0.6%) | |
| Other | 16 (8.3%) | 18 (10.1%) | |
| Chronically sun-exposed | | | |
| anatomic site^{#} | | | |
| No | 95 (49.7%) | 105 (42.7%) | p = 0.14 |
| Yes | 96 (50.3%) | 141 (57.3%) | |
| Received any | | | |
| non-surgical therapy | | | |
| No | 153 (79.7%) | 201 (83.1%) | p = 0.37 |
| Yes | 39 (20.3%) | 41 (16.9%) | |
| Microsatellitosis | | | |
| Absent | 149 (77.6%) | — | N/A |
| Present | 43 (22.4%) | — | |
| Positive sentinel lymph | | | |
| node biopsy | | | |
| No | — | 211 (87.6%) | N/A |
| Yes | — | 30 (12.4%) | |

*Numbers may not sum to total due to missing values, percents may not sum to 100% due to rounding.
^{†}Significant at p < 0.05
^{#}Anatomic location was dichotomized as chronically sun exposed (face, scalp, neck, arms, legs and non-acral lentiginous lesions of hands and feet) and non-chronically exposed (chest, back, abdomen, groin, hand and foot acral lentiginous lesions).

Clinicopathologic Correlates of Candidate Marker Expression

Thirty-eight unique protein markers were assayed by AQUA on the Discovery cohort (n=192) and, for comparison, the Metastatic Series (n=299). Exclusion of individual tumors due to random failure for individual histospots as well as attrition of samples due to exhaustion of the arrayed tumor core resulted in fewer than 100% of tumor samples available for analysis from each assay. Only the subset of 20 markers with missingness completely at random was included in subsequent analyses.

Associations between levels of protein expression and tumor progression were evaluated by the Mann-Whitney U test (Supplemental Table 2). Following adjustment for multiple comparisons, levels of fibronectin, Ki-67, and p21^{WAF1}, as well as the ratios for both ATF2 and p16^{INK4A} were significantly elevated whereas Hey1, HDM2, N-cadherin, nuclear p16^{INK4A}, and non-nuclear ATF2 were significantly decreased among the metastases compared to the primary tumors (p≦0.0025).

SUPPLEMENTARY TABLE 2
Marker expression levels among primary and
metastatic lesions*

| | | Metastatic | |
|---|---|---|---|
| | Primary tumors | tumors mean ± | Mann- |
| | mean ± SD | SD | Whitney U |
| Target* | (n = 192) | (n = 299) | p-value |
| α-catenin | 11.53 ± 5.86 | 13.10 ± 6.72 | p = 0.02^{†} |
| Annexin-1/Lipocortin-1 | 40.48 ± 20.89 | 42.09 ± 25.87 | p = 0.54 |
| ATF-2 - non-nuclear | 65.57 ± 39.33 | 46.87 ± 35.65^{#} | p < 0.0001^{‡} |
| compartment | | | |
| ATF-2 - In (non-nuclear/ | 1.07 ± 0.58 | 1.40 ± 0.75^{#} | p < 0.0001^{‡} |
| nuclear compartments) | | | |
| β-catenin | 48.28 ± 15.75 | 43.18 ± 13.18 | p = 0.002^{†} |
| Fibronectin | 47.49 ± 13.74 | 64.10 ± 24.45^{#} | p < 0.0001^{‡} |
| Hairy/Enhancer of split- | 58.93 ± 23.89 | 48.23 ± 22.93 | p < 0.0001^{‡} |
| related-1 | | | |
| Human double-minute- | 65.80 ± 21.53 | 52.36 ± 20.13^{#} | p < 0.0001^{‡} |
| 2 - nuclear compartment | | | |
| Integrin-linked kinase | 42.95 ± 13.60 | 44.17 ± 12.91 | p = 0.06 |
| Ki-67 - nuclear | 18.77 ± 6.84 | 23.03 ± 9.63 | p < 0.0001^{‡} |
| compartment | | | |
| Matrix | 36.58 ± 18.54 | 33.69 ± 22.61 | p = 0.008^{†} |
| metalloproteinase-1 | | | |
| N-cadherin | 19.48 ± 15.33 | 11.82 ± 11.42 | p < 0.0001^{‡} |
| Osteonectin/SPARC | 19.89 ± 14.42 | 20.14 ± 17.12 | p = 0.10 |
| p16/INK4A - nuclear | 32.34 ± 26.69 | 24.85 ± 19.00 | p = 0.0006^{‡} |
| compartment | | | |
| p16/INK4A - in (non- | −0.15 ± 0.32 | −0.07 ± 0.25 | p < 0.0001^{‡} |
| nuclear/nuclear | | | |
| compartments) | | | |
| p21/WAF1/CIP1 - | 17.76 ± 9.06 | 24.14 ± 13.60 | p < 0.0001^{‡} |
| nuclear compartment | | | |
| p27/KIP1 - nuclear | 44.64 ± 21.82 | 46.23 ± 21.97 | p = 0.33 |
| compartment | | | |
| p27/KIP1 - in(non- | −0.33 ± 0.26 | −0.35 ± 0.28 | p = 0.30 |
| nuclear/nuclear | | | |
| compartments) | | | |
| P-cadherin | 35.71 ± 7.11 | 33.69 ± 6.08 | p = 0.007^{†} |
| Tenascin-C | 26.12 ± 21.24 | 33.53 ± 29.80 | p = 0.28 |

*Each target considers the AQUA score under the entire tumor mask, unless otherwise indicated
^{†}Significant at p = 0.05
^{‡}Significant at the Bonferroni-adjusted p-value of p = 0.0025.
^{#}Assay of Fibronectin, HDM2 and ATF-2 was as restricted to the subset of 198 metastases collected during 1959-1994.

To determine the independent crude and adjusted effects of each marker on melanoma-specific mortality, the AQUA scores or calculated ratios were divided into quartiles and the hazard ratios and associated p-values calculated using Cox proportional hazards modeling (Supplemental Table 4). Using these cut-points, five markers, one that increased risk with increasing value (p16^{INK4A} ratio, p=0.04) and four that decreased risk with increased value (ATF2, p=0.001; β-catenin, p=0.04; N-cadherin, p=0.001; p16^{INK4A}, p=0.047) were significant at p<0.05 on univariate analysis but only 2, ATF2 and N-cadherin, remained significant following adjustment for multiple comparisons (p≦0.0025).

SUPPLEMENTARY TABLE 4
Individual marker associations with melanoma-specific mortality

| | Univariate | | Multivariable | |
|---|---|---|---|---|
| Target | HR (95% CI) | p-value^{†} | HR (95% CI)* | p-value |
| α-catenin | | | | |
| Quartile 1 (AQUA score 2.56-6.82) | 1.00 | p = 0.43 | 1.00 | p = 0.01^{#} |
| Quartile 2 (AQUA score 6.83-10.95) | 1.21 (0.66-2.24) | | 1.28 (0.68-2.42) | |
| Quartile 3 (AQUA score 10.96-14.77) | 1.01 (0.54-1.89) | | 0.50 (0.25-0.99) | |
| Quartile 4 (AQUA score 14.78-34.31) | 0.69 (0.34-1.40) | | 0.43 (0.19-0.97) | |
| Annexin-1/Lipocortin-1 | | | | |
| Quartile 1 (AQUA score 4.53-21.89) | 1.00 | p = 0.28 | 1.00 | p = 0.75 |
| Quartile 2 (AQUA score 21.90-41.47) | 1.02 (0.50-2.06) | | 1.04 (0.50-2.18) | |
| Quartile 3 (AQUA score 41.48-53.79) | 1.38 (0.71-2.68) | | 1.36 (0.67-2.77) | |
| Quartile 4 (AQUA score 53.80-103.66) | 1.75 (0.90-3.40) | | 0.97 (0.47-1.99) | |
| ATF-2 - non-nuclear compartment | | | | |
| Quartile 1 (AQUA score 13.08-36.11) | 1.00 | p = 0.001^{‡} | 1.00 | p = 0.25 |
| Quartile 2 (AQUA score 36.12-59.10) | 0.74 (0.42-1.29) | | 0.89 (0.48-1.63) | |
| Quartile 3 (AQUA score 59.11-87.23) | 0.62 (0.35-1.10) | | 0.73 (0.39-1.37) | |
| Quartile 4 (AQUA score 87.24-244.30) | 0.25 (0.12-0.54) | | 0.47 (0.21-1.04) | |
| ATF-2 - in (non-nuclear/nuclear compartments) | | | | |
| Quartile 1 (Ratio −1.33-−0.14) | 1.00 | p = 0.20 | 1.00 | p = 0.79 |
| Quartile 2 (Ratio −0.13-+0.04) | 0.57 (0.30-1.08) | | 0.95 (0.48-1.90) | |
| Quartile 3 (Ratio +0.05-+0.34) | 0.61 (0.33-1.12) | | 1.23 (0.62-2.46) | |
| Quartile 4 (Ratio +0.35-+2.38) | 0.58 (0.32-1.05) | | 0.87 (0.45-1.66) | |
| β-catenin | | | | |
| Quartile 1 (AQUA score 9.46-37.83) | 1.00 | p = 0.04^{#} | 1.00 | p = 0.04^{#} |
| Quartile 2 (AQUA score 37.84-45.59) | 0.52 (0.28-0.97) | | 0.40 (0.20-0.83) | |
| Quartile 3 (AQUA score 45.60-55.94) | 0.64 (0.34-1.18) | | 0.86 (0.44-1.67) | |
| Quartile 4 (AQUA score 55.95-104.05) | 0.40 (0.20-0.79) | | 0.45 (0.21-0.96) | |
| Fibronectin | | | | |
| Quartile 1 (AQUA score 23.00-37.06) | 1.00 | p = 0.17 | 1.00 | p = 0.33 |
| Quartile 2 (AQUA score 37.07-45.67) | 1.24 (0.64-2.41) | | 1.05 (0.50-2.22) | |
| Quartile 3 (AQUA score 45.68-57.01) | 0.66 (0.31-1.43) | | 0.70 (0.31-1.59) | |
| Quartile 4 (AQUA score 57.02-93.87) | 1.43 (0.74-2.76) | | 1.43 (0.68-2.98) | |
| Hairy/Enhancer of Split-related-1 | | | | |
| Quartile 1 (AQUA score 6.09-43.14) | 1.00 | p = 0.31 | 1.00 | p = 0.22 |
| Quartile 2 (AQUA score 43.15-54.77) | 1.37 (0.72-2.59) | | 1.13 (0.56-2.26) | |
| Quartile 3 (AQUA score 54.78-74.04) | 0.76 (0.37-1.54) | | 0.54 (0.25-1.16) | |
| Quartile 4 (AQUA score 74.05-173.42) | 1.26 (0.65-2.45) | | 1.07 (0.51-2.23) | |
| Human double-minute-2 - nuclear compartment | | | | |
| Quartile 1 (AQUA score 21.84-50.09) | 1.00 | p = 0.34 | 1.00 | p = 0.24 |
| Quartile 2 (AQUA score 50.10-62.04) | 0.65 (0.33-1.28) | | 0.79 (0.37-1.68) | |
| Quartile 3 (AQUA score 62.05-76.06) | 0.97 (0.51-1.85) | | 1.24 (0.64-2.42) | |
| Quartile 4 (AQUA score 76.07-145.79) | 0.60 (0.29-1.22) | | 0.57 (0.26-1.24) | |
| Integrin-linked kinase | | | | |
| Quartile 1 (AQUA score 15.82-33.17) | 1.00 | p = 0.31 | 1.00 | p = 0.29 |
| Quartile 2 (AQUA score 33.18-40.70) | 0.78 (0.42-1.46) | | 0.54 (0.28-1.05) | |
| Quartile 3 (AQUA score 40.71-50.10) | 0.70 (0.36-1.35) | | 0.75 (0.37-1.53) | |
| Quartile 4 (AQUA score 50.11-96.77) | 0.51 (0.25-1.06) | | 0.62 (0.28-1.34) | |
| Ki-67 - nuclear compartment | | | | |
| Quartile 1 (AQUA score 4.25-13.83) | 1.00 | p = 0.66 | 1.00 | p = 0.21 |
| Quartile 2 (AQUA score 13.84-18.08) | 1.12 (0.60-2.09) | | 0.91 (0.47-1.77) | |
| Quartile 3 (AQUA score 18.09-22.67) | 0.86 (0.42-1.75) | | 0.47 (0.21-1.05) | |
| Quartile 4 (AQUA score 22.68-40.24) | 1.32 (0.69-2.52) | | 0.91 (0.45-1.83) | |
| Matrix metalloproteinase-1 | | | | |
| Quartile 1 (AQUA score 9.49-21.57) | 1.00 | p = 0.46 | 1.00 | p = 0.66 |
| Quartile 2 (AQUA score 21.58-33.09) | 1.51 (0.75-3.07) | | 0.96 (0.44-2.09) | |
| Quartile 3 (AQUA score 33.10-47.86) | 1.16 (0.56-2.39) | | 0.97 (0.44-2.16) | |
| Quartile 4 (AQUA score 47.87-91.51) | 1.64 (0.82-3.26) | | 1.45 (0.68-3.09) | |
| N-cadherin | | | | |
| Quartile 1 (AQUA score 4.17-8.30) | 1.00 | p = 0.001^{‡} | 1.00 | p = 0.06 |
| Quartile 2 (AQUA score 8.31-14.54) | 0.83 (0.47-1.48) | | 0.54 (0.27-1.06) | |
| Quartile 3 (AQUA score 14.54-24.83) | 0.37 (0.18-0.76) | | 0.45 (0.21-0.98) | |
| Quartile 4 (AQUA score 24.84-77.09) | 0.32 (0.16-0.66) | | 0.38 (0.18-0.82) | |
| Osteonectin/SPARC | | | | |
| Quartile 1 (AQUA score 4.92-9.69) | 1.00 | p = 0.40 | 1.00 | p = 0.23 |
| Quartile 2 (AQUA score 9.70-15.45) | 1.78 (0.89-3.55) | | 1.83 (0.89-3.76) | |
| Quartile 3 (AQUA score 15.46-23.45) | 1.42 (0.71-2.86) | | 0.92 (0.45-1.92) | |
| Quartile 4 (AQUA score 23.46-68.19) | 1.53 (0.78-3.01) | | 1.25 (0.61-2.57) | |
| p16/INK4A - nuclear compartment | | | | |
| Quartile 1 (AQUA score 4.18-15.14) | 1.00 | p = 0.047^{#} | 1.00 | p = 0.04^{#} |
| Quartile 2 (AQUA score 15.15-22.69) | 0.48 (0.27-0.89) | | 0.46 (0.27-0.88) | |
| Quartile 3 (AQUA score 22.70-39.78) | 0.56 (0.31-1.01) | | 0.42 (0.22-0.81) | |
| Quartile 4 (AQUA score 39.79-158.00) | 0.48 (0.26-0.88) | | 0.60 (0.32-1.15) | |
| p16/INK4A - in (non-nuclear/nuclear compartments) | | | | |
| Quartile 1 (Ratio −0.93-−0.35) | 1.00 | p = 0.04^{#} | 1.00 | p = 0.15 |
| Quartile 2 (Ratio −0.34-−0.18) | 0.73 (0.36-1.50) | | 0.68 (0.32-1.47) | |
| Quartile 3 (Ratio −0.17-+0.03) | 1.45 (0.78-2.71) | | 1.39 (0.72-2.71) | |
| Quartile 4 (Ratio +0.04-+1.24) | 1.75 (0.96-3.20) | | 1.31 (0.68-2.55) | |
| p21/WAF1/CIP1 - nuclear compartment | | | | |
| Quartile 1 (AQUA score 7.49-12.18) | 1.00 | p = 0.24 | 1.00 | p = 0.24 |
| Quartile 2 (AQUA score 12.19-15.08) | 0.95 (0.52-1.73) | | 0.84 (0.44-1.58) | |
| Quartile 3 (AQUA score 15.09-21.48) | 0.66 (0.34-1.27) | | 0.49 (0.24-1.00) | |
| Quartile 4 (AQUA score 21.49-76.02) | 1.28 (0.71-2.31) | | 0.74 (0.38-1.46) | |
| p27/KIP1 - nuclear compartment | | | | |
| Quartile 1 (AQUA score 10.27-28.95) | 1.00 | p = 0.67 | 1.00 | p = 0.046^{#} |
| Quartile 2 (AQUA score 28.96-39.82) | 1.41 (0.77-2.62) | | 2.58 (1.32-4.87) | |
| Quartile 3 (AQUA score 39.83-53.99) | 1.02 (0.53-1.96) | | 1.27 (0.62-2.63) | |
| Quartile 4 (AQUA score 54.00-144.49) | 1.10 (0.59-2.07) | | 1.53 (0.75-3.11) | |
| p27/KIP1 - in (non-nuclear/nuclear compartments) | | | | |
| Quartile 1 (Ratio −1.38-−0.49) | 1.00 | p = 0.23 | 1.00 | p = 0.36 |
| Quartile 2 (Ratio −0.48-−0.31) | 0.72 (0.37-1.41) | | 0.54 (0.27-1.08) | |
| Quartile 3 (Ratio −0.30-−0.13) | 0.88 (0.47-1.65) | | 0.63 (0.32-1.24) | |
| Quartile 4 (Ratio −0.12-+0.22) | 1.39 (0.76-2.55) | | 0.69 (0.35-1.34) | |
| P-cadherin | | | | |
| Quartile 1 (AQUA score 20.02-30.95) | 1.00 | p = 0.70 | 1.00 | p = 0.49 |
| Quartile 2 (AQUA score 30.96-35.24) | 0.75 (0.40-1.42) | | 1.14 (0.56-2.33) | |
| Quartile 3 (AQUA score 35.25-39.57) | 0.72 (0.37-1.39) | | 0.92 (0.44-1.94) | |
| Quartile 4 (AQUA score 39.58-60.85) | 0.95 (0.50-1.81) | | 1.62 (0.81-3.24) | |
| Tenascin-C | | | | |
| Quartile 1 (AQUA score 6.54-12.32) | 1.00 | p = 0.30 | 1.00 | p = 0.046^{#} |
| Quartile 2 (AQUA score 12.33-18.65) | 0.86 (0.43-1.69) | | 0.65 (0.30-1.37) | |
| Quartile 3 (AQUA score 18.66-33.95) | 0.71 (0.35-1.43) | | 0.92 (0.40-2.09) | |
| Quartile 4 (AQUA score 33.96-142.89) | 1.32 (0.70-2.51) | | 1.81 (0.88-3.74) | |

*Adjusted for age at diagnosis, gender, Breslow thickness (mm), stage at diagnosis, presence of microsatellitosis, sun exposure to anatomic site and receipt of non-surgical therapy.
^{†}Univariate and multivariable p-values were calculated according to the likelihood ratio test method.
^{#}Significant at p ≦ 0.05.
^{‡}Significant at Bonferroni-adjusted p ≦ 0.0025.

Multivariable Cox proportional hazards modeling included adjustment for age at diagnosis, gender, Breslow thickness (mm), stage at diagnosis, presence of microsatellitosis, sun exposure to anatomic site and receipt of systemic therapy. Two of the five markers significant on univariate analysis, (β-catenin (p=0.04), p16^{INK4A} (p=0.04) retained both their significance at p<0.05 and directionality of effect following adjustment for clinicopathologic parameters. Three additional markers that were not significant on crude analysis became significant at p<0.05 on multivariable analysis (α-catenin, p27/KIP1 and tenascin-C).

Constructing a Genetic Algorithm-based Multi-Marker Prognostic Model

Since the power of multiplexed biomarker assays is thought to be greater than that obtainable with any single marker, we sought to identify a robust prognostic indicator by combining information from all 20 available markers, regardless of whether a significant independent association with progression or prognosis was obtained, using genetic algorithms. Our selected model, obtained in each of the 5 independent iterations, yielded a log-rank chi-square of 24.27 (p=1.5×10⁻⁶) and consisted of the following 5 markers and associated cut-points: ATF2 ratio >−0.052, β-catenin>38.68, fibronectin≦57.93, p16^{INK4A} ratio≦−0.083, and p21^{WAF1}>12.98.

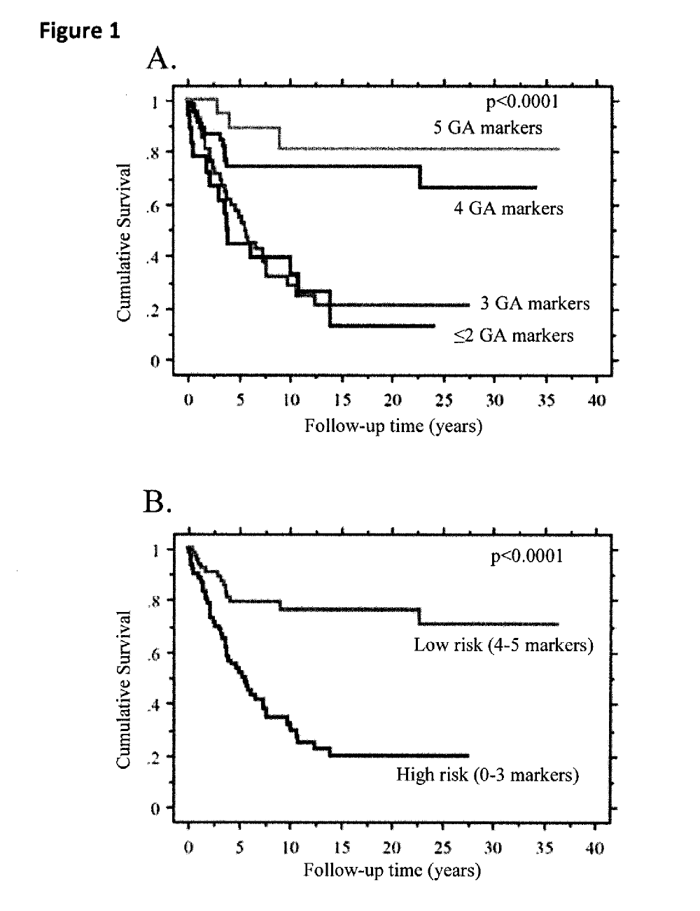

The Kaplan-Meier curves for the 4 classes obtained from the genetic algorithm are presented (FIG. 1a). Based on the similar survival experiences of the groupings with ≦2 or 3 conditions and those with 4 or 5 conditions, we further simplified our model to 2 states: a low-risk state with 4 or 5 marker conditions being met and a high-risk state with fewer than 4 marker conditions being met (FIG. 1b). Crude and multivariable survival estimates were calculated for the multi-marker predictor and the clinicopathologic covariates using Cox proportional hazards modeling (Table 2). In our final multivariable model, the high-risk group demonstrated a nearly 3-fold increased risk of mortality (p=0.002) over those with low risk. Other variables remaining significant in the multivariable model included stage at diagnosis and receipt of non-surgical therapy (p≦0.01) Breslow thickness trending towards significance (p=0.06).

TABLE 2
Crude and multivariable-adjusted melanoma-specific mortality hazard ratios (95% CI)
for the genetic algorithm-based multi-marker predictor in the Yale Melanoma Discovery cohort

| | Univariate | | Multivariable | |
|---|---|---|---|---|
| Parameter | HR (95% CI) | p-value^{†} | HR (95% CI) | p-value |
| Genetic algorithm-based predictor | | | | |
| Low-risk group (4 or 5 conditions met) | 1.00 | p < 0.0001* | 1.00 | p = 0.002* |
| High-risk group (<4 conditions met) | 3.88 (2.16-6.94) | | 2.84 (1.46-5.49) | |
| Breslow thickness (mm) | 1.28 (1.14-1.43) | p < 0.0001* | 1.14 (0.99-1.31) | p = 0.06 |
| Age at diagnosis (yrs) | 1.01 (0.99-1.03) | p = 0.41 | 1.01 (0.99-1.03) | p = 0.39 |
| Gender | | | | |
| Male | 1.00 | p = 0.14 | 1.00 | p = 0.14 |
| Female | 0.68 (0.41-1.14) | | 0.66 (0.38-1.14) | |
| Stage at diagnosis | | | | |
| Localized | 1.00 | p = 0.0006* | 1.00 | p = 0.0002* |
| Regional spread | 3.54 (1.72-7.30) | | 4.67 (2.08-10.47) | |
| Distant metastases | 5.05 (2.35-10.94) | p < 0.0001* | 3.32 (1.31-8.39) | p = 0.01* |
| Chronically sun-exposed anatomic site | | | | |
| No | 1.00 | p = 0.03* | 1.00 | p = 0.24 |
| Yes | 0.56 (0.33-0.95) | | 0.70 (0.39-1.26) | |
| Microsatellitosis | | | | |
| Absent | 1.00 | p = 0.047* | 1.00 | p = 0.63 |
| Present | 1.73 (1.01-2.96) | | 1.16 (0.64-2.11) | |
| Receipt of non-surgical therapy | | | | |
| No | 1.00 | p = 0.0005* | 1.00 | p = 0.008* |
| Yes | 2.54 (1.50-4.30) | | 2.31 (1.25-4.26) | |

^{†}p-values calculated according to the Wald method
*Significant at p ≦ 0.05

Assessment of Multi-Marker Model Reproducibility in the Validation Cohort

To determine the prognostic breadth and strength of our genetic algorithm-based multi-marker predictor, we performed the assay on the independent Validation TMA, normalizing the 2 builds as described. Complete AQUA data was obtained for 226 of the 246 eligible individuals with 76 individuals (33.6%) meeting criteria for the low-risk group and 150 (66.4%) belonging to the high-risk group. Notably, our predictor was independent of both Breslow thickness (p=0.41) and sentinel lymph node status (p=0.52) (Table 3). Our predictor trended towards, but did not achieve, significance for melanoma-specific mortality in univariate analysis (Table 4). Yet, multivariable modeling that adjusted for Breslow thickness, age at diagnosis, anatomic site, sentinel lymph node biopsy status and receipt of non-surgical therapy revealed a significantly increased melanoma-specific mortality for the high-risk group (adjusted HR=2.72, 95% CI=1.12-6.58; p=0,027) (Table 4), consistent with the possibility of negative confounding by clinicopathologic parameters in the validation set. Our predictor is independent of sentinel lymph node status and the interaction between multi-marker assignment and sentinel lymph node status was not significant (p=0.78).

TABLE 3
Bivariate associations between the genetic algorithm-derived prognostic
indicator and clinicopathologic correlates of melanoma outcome for the
Yale Melanoma Validation cohort.

| | | High-risk | |
|---|---|---|---|
| | Low-risk group | group | |
| Parameter | (n = 76)* | (n = 150) | p-value |
| Breslow thickness (mm) | 1.86 ± 1.73 | 2.08 ± 1.89 | p = 0.41 |
| Age at diagnosis (yrs) | 57.17 ± 15.66 | 61.14 ± 16.57 | p = 0.08 |
| Gender | | | |
| Male | 41 (54.0%) | 97 (64.7%) | p = 0.12 |
| Female | 35 (46.1%) | 53 (35.3%) | |
| Ulceration | | | |
| Absent | 63 (82.9%) | 117 (78.0%) | p = 0.39 |
| Present | 13 (17.1%) | 33 (22.0%) | |
| Tumor-infiltrating | | | |
| lymphocytes | | | |
| Non-brisk | 64 (84.2%) | 126 (84.6%) | p = 0.94 |
| Brisk | 12 (15.8%) | 23 (15.4%) | |
| Histologic subtype | | | |
| Superficial spreading | 45 (76.3%) | 76 (72.4%) | p = 0.43 |
| Nodular | 7 (11.9%) | 16 (15.2%) | |
| Lentigo maligna | 2 (3.4%) | 1 (1.0%) | |
| Acral lentiginous | 1 (1.7%) | 0 (0.0%) | |
| Other | 4 (6.8%) | 12 (11.4%) | |
| Chronically sun-exposed | | | |
| anatomic site | | | |
| No | 33 (43.4%) | 65 (43.3%) | p = 0.99 |
| Yes | 43 (56.6%) | 85 (56.7%) | |
| Received any | | | |
| non-surgical therapy | | | |
| No | 60 (80.0%) | 123 (83.7%) | p = 0.50 |
| Yes | 15 (20.0%) | 24 (16.3%) | |
| Sentinel lymph node biopsy | | | |
| Negative | 64 (85.3%) | 129 (88.4%) | p = 0.52 |
| Positive | 11 (14.7%) | 17 (11.6%) | |

*Numbers may not sum to total due to missing values, percents may not sum to 100% due to rounding.

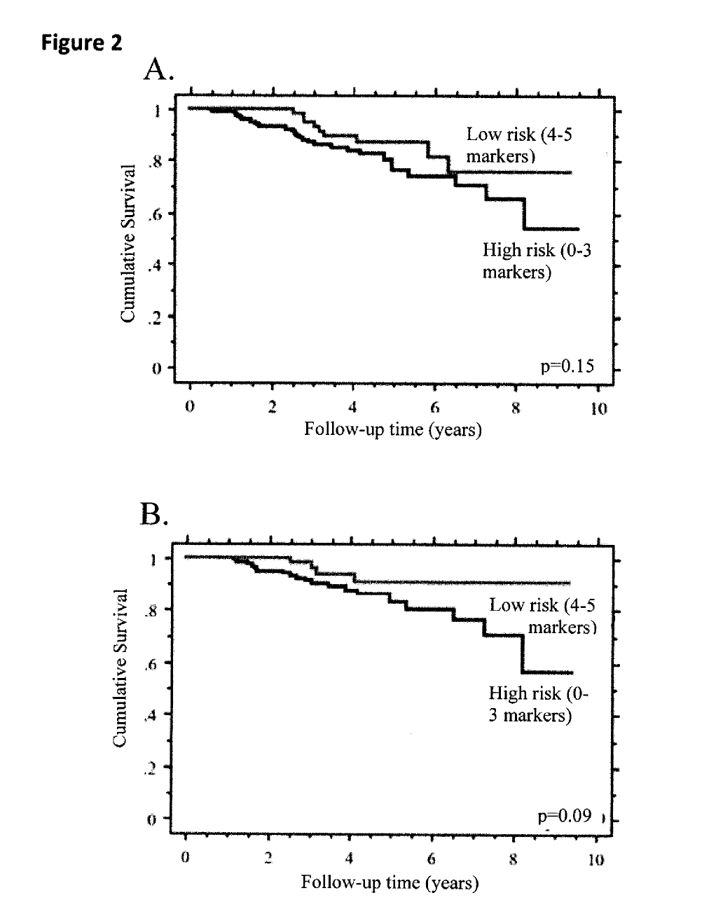

Although the multivariate analysis of the validation set is statistically significant (Table 4) the Kaplan-Meier analysis of the validation set is not (FIG. 2A) most likely due to the confounding effect of non-uniform treatment. McShane at al in the REMARK guidelines point out the value of the multivariate analysis over the log rank assessment done on the Kaplin-Meier data. This work is an example of the multivariate analysis adjusting for confounding to show significance, as anticipated by the REMARK criteria However, the Kaplin-Meier plot is shown to help convey the data in a more simple form related to the envisioned utility of the test in sentinel node negative patients. In this population, the high-risk group has only a 60% ten-year survival compared to a ten-year survival of over 90% in the low-risk group (FIG. 2B, log rank p=0.09).

TABLE 4
Crude and multivariable-adjusted melanoma-specific mortality hazard ratios (95% CI)
for the genetic algorithm-based multi-marker predictor in the Yale Melanoma Validation cohort

| | Univariate | | Multivariable | |
|---|---|---|---|---|
| Parameter | HR (95% CI) | p-value^{†} | HR (95% CI) | p-value |
| Genetic algorithm-based predictor | | | | |
| Low-risk group (4 or 5 conditions met) | 1.00 | p = 0.14 | 1.00 | p = 0.027* |
| High-risk group (<4 conditions met) | 1.75 (0.83-3.72) | | 2.72 (1.12-6.58) | |
| Breslow thickness (mm) | 1.20 (1.11-1.31) | p < 0.0001 | 1.14 (1.01-1.29) | p = 0.029* |
| Age at diagnosis (yrs) | 1.03 (1.00-1.05) | p = 0.027 | 1.04 (1.01-1.07) | p = 0.007* |
| Gender | | | | |
| Male | 1.00 | p = 0.07 | 1.00 | p = 0.10 |
| Female | 0.51 (0.25-1.06) | | 0.52 (0.24-1.14) | |
| Chronically sun-exposed anatomic site | | | | |
| No | 1.00 | p = 0.20 | 1.00 | p = 0.11 |
| Yes | 1.55 (0.79-3.04) | | 1.96 (0.87-4.44) | |
| Sentinel lymph node biopsy status | | | | |
| Negative | 1.00 | p < 0.0001 | 1.00 | p = 0.017* |
| Positive | 4.41 (2.23-8.71) | | 2.78 (1.20-6.47) | |
| Receipt of non-surgical therapy | | | | |
| No | 1.00 | p < 0.0001 | 1.00 | p = 0.0001* |
| Yes | 7.09 (3.60-13.96) | | 4.65 (2.11-10.24) | |

^{†}p-values calculated according to the Wald method
*Significant at p ≦ 0.05

Discussion

Over the last few years, multi-marker molecular models have been constructed to supplement available clinicopathologic parameters for refining prognosis in some tumor types. Here, we report on a multi-marker melanoma prognostic assay with potential for translation into the clinic that may be especially useful for identifying the subset of Stage II melanoma patients most appropriate for supplemental therapy. Presently, up to 40% of patients with Stage IIA-IIB melanoma will die of their disease within 10 years of diagnosis Due to the poor risk-benefit ratio and toxicity of current adjuvant therapy regimens², it is not often given in this population. We believe there is a significant clinical need to stratify this population at the time of diagnosis, into a subset of Stage II patients with the highest risk for recurrence and a lower risk group. The goal of this stratification, using the test described here, would be to offer adjuvant intervention or at least aggressive follow-up screening to high risk stage II patients. We believe this would improve the overall survival of these vulnerable patients without exposing the remaining patients to the risk of excessive toxicity and thus this test has the potential to alter the standard of care for management of melanoma.

To our knowledge, only one other prognostic multi-marker molecular classifier for primary melanoma has been described specifying a 254-gene classifier obtained from differential mRNA expression profiling on a series of 83 snap-frozen samples¹⁷. Although protein expression by IHC was confirmed for the 23-gene subset with commercially-available antibodies, the authors only reported on their marginal univariate and multivariable prognostic relationships. While this study is valuable, to date, the multi-marker classifier has not been validated on a second population. Additional molecular classifiers of melanoma phenotype that integrate either somatic mutation (e.g., Viros, 2008¹⁸) or gene expression information (e.g., Bittner, 2000¹⁹) have been reported but have not been evaluated for prognostic relevance. Efforts that use hierarchical clustering, which is valuable for classification, suffer from the inabilities to both calculate error associated with a clustering run and prospectively assign new patients to existing clusters without re-executing the clustering which risks reorganizing cluster assignment. Assignment of new cases according to our genetic algorithm profile, as demonstrated in our validation strategy, only requires simultaneous AQUA analysis of selected reference standards.

Assignment to the “low-risk” class requires elevated levels of overall β-catenin and nuclear p21^{WAF1}, decreased levels of fibronectin, and distributions that favor nuclear concentration for p16^{INK4A} but cytoplasmic concentration for ATF2. Each of these assignments is consistent with the previous literature for melanoma. Our data, as well as that from others^{20,21} support that increased nuclear p16^{INK4A} expression, significantly improves melanoma prognosis in multivariable modeling, consistent with its role in cell cycle inhibition²². Although specific cytoplasmic p16^{INK4A} expression has been confirmed by multiple high-resolution imaging technologies^{23,24,} little is known about its functional role or prognostic implications. Our data suggest that a ratio that favors nuclear abundance contributes to improved cell cycle control. Similar rationale can be suggested for elevated nuclear p21^{WAF1}, however neither we nor others have shown a significant effect for the marginal effects of nuclear p21^{WAF1} on univariate^{25,26} or multivariable^{20,27} analysis. The requirement for a higher proportion of cytoplasmic ATF2 is supported by the observation that although ATF2 possesses both nuclear export and nuclear localization signals and shuttles between both locations, nuclear heterodimerization with c-Jun and subsequent phosphorylation of both subunits by MAP kinases are required for transcriptional activation activity^{28,29}. Although we did not distinguish between membranous cadherin-associated and cytoplasmic/nuclear Wnt-signaling-associated β-catenin, our association between improved prognosis and elevated β-catenin is consistent with others^{30,31}. Finally, our requirement for reduced fibronectin supports both tissue- and cell-based observations that increased tumor-derived expression facilitates melanoma cell invasion and metastasis³²⁻³⁴.

This work suffers from a number of limitations. Perhaps the most significant limitation is the relatively limited set of available markers eligible for our analysis. Unlike nucleic acid arrays where tens of thousands of genes can be interrogated in each experiment, we can only assess one gene product at a time (although we have the advantage of assessing hundreds of patients per experiment). Furthermore, more than half of the markers initially considered for this study were ultimately eliminated from our genetic algorithm due to preferential attrition of longer-surviving (typically thinner) melanomas due to exhaustion of their tissue cores with higher cuts of the TMA. Future replication of these results on parallel blocks of the Discovery TMA may both fill gaps and also provide useful information regarding heterogeneity of marker expression. Although we selected a broad range of candidate targets, the inherent limitation of the candidate gene approach omitted sufficient markers from some cancer progression pathways such as evading apoptosis, sustained angiogenesis or insensitivity to anti-growth signals³⁵. Additionally, several proteins previously shown by others to have significant independent marginal associations with melanoma outcome, such as MMP-2^{36,37}, osteopontin³⁸, MCAM/MUC18^{39,40} and AIB-1⁴¹ were not assayed (in some cases due to antibody validation failure). Another theoretical weakness of this approach is that our genetic algorithm equally weighted each protein's individual contribution. This is in contrast to a commercially available breast cancer diagnostic (Oncotype DX) where individual marker contribution is weighted according to its relative marginal contribution to the overall model⁴². The genetic algorithm approach risks bias in group assignment should the presence or absence of one specific marker disproportionately drive assignment into one of the algorithm states. However, as shown above, we found that this bias did not occur in our discovery phase.

Strengths of our approach include the use of equally large and robust, yet completely independent, training and validation study populations as well as choice of a computational, method that supports the prospective evaluation of new patients according to its calculated criteria. Given that we were able to replicate a significant, independent association between our multi-marker prognostic assay and melanoma-specific mortality after adjustment for relevant clinicopathologic covariates in our independently collected validation set, we believe this data could support the use of this test to assist management of patients with sentinel node negative melanoma. For example, a negative sentinel node patient with a high risk test result might prompt a patient to choose adjuvant therapy. While the data on the efficacy of adjuvant interferon is controversial⁴³, other adjuvant therapies such as ipilimumab and vaccine therapies are currently under investigation, and these studies typically include only stage III patients. However, high risk stage II patients identified by improved prognostic assays such as this, should also be considered for these studies. Prospective validation is planned in a broader geographic constituency to determine if this method should become part of the routine work up for patients with malignant melanoma.

Part 3

Supplemental Methods:

Patients and Tumor Samples

Stage at diagnosis (localized, regional and distant) and anatomic location were obtained from the surgical report. Receipt of non-surgical therapy referred to administration of cytotoxic chemotherapy, immunomodulators or radiotherapy either in the adjuvant setting or following clinical recurrence. For each cohort, a single investigator reviewed all slides to reconfirm the diagnosis of melanoma and to determine Breslow thickness, Clark level of invasion, histopathologic subtype, and the presence of ulceration, microsatellitosis and tumor-infiltrating lymphocytes.

Tissue Microarray Construction, Immunohistochemistry and Automated Quantitative Image Analysis

Internal positive and negative controls for both the Discovery and Validation TMAs comprised of single cores from FFPE preparations of 15 melanocytic cell lines for which protein expression is verified by Western blot¹. The cell lines included BHP 18-21, Mel 501, Mel 624, Mel 888, Mel 928, Mel 1241, Mel 1335, MM127, MNT-1, SK23, YUMAC2, YUMOR, YUSAC2, YUSIT1, YUGEN8, and normal melanocytes derived from neonatal foreskin. Sections (5 μm) were cut from the TMA master using a tissue microtome, transferred to glass slides using a UV cross-linkable tape transfer system (Instrumedics, St. Louis, Mo.), dipped in paraffin and stored in a nitrogen chamber to prevent antigen degeneration before staining(44).

Slides were deparaffinized using 2 xylene exchanges followed by rehydration through an ethanol gradient and washed with tris-buffered saline (TBS). Antigen retrieval was performed by boiling the slides in a sealed pressure cooker containing 6.5 mmol/L sodium citrate, pH 6.0 (except for ATF2 and HDM2 where EDTA, pH 7.5 was used) for 15 minutes. Next, the slides were immersed in absolute methanol containing 0.75% hydrogen peroxide for 30 minutes to neutralize endogenous peroxidase activity, followed by incubation for 30 minutes in 0.3% bovine serum albumin (BSA) dissolved in 1 mol/L TBS (pH 8.0) to block non-specific binding.

Fluorescence-based immunohistochemical staining was performed by multiplexing a primary antibody directed against a candidate protein with an S100B antibody of a complementary species (DAKO (Carpenteria, Calif.) rabbit anti-S100B polyclonal at 1:600 or Biogenex (San Ramon, Calif.) mouse anti-S100B monoclonal at 1:100), the latter to distinguish melanoma from the surrounding stroma in the absence of counterstain. The selection of protein candidates and their corresponding antibody reagents are presented in Supplemental Table 1. External negative controls were obtained by omitting the target protein primary antibody. Primary antibodies were incubated at 4° C. overnight. The secondary antibodies, Alexa-546-conjugated goat antibody directed against the anti-S100 antibody (anti-mouse or anti-rabbit; 1:200, Molecular Probes, Eugene, Oreg.) diluted into Envision, an HRP-tagged polymer, directed against the protein candidate (neat; DAKO) were applied for 1 hour at room temperature. To visualize the nuclei, 4′,6-diamidino-2-phenylindole (DAPI, 1:100) was included with the secondary antibodies. Finally, a 10-minute Cy5-tyramide (Perkin Elmer Life Sciences, Wellesley, Mass.) incubation labeled the target. The slides were mounted with 0.6% n-propyl galleate antifade reagent, sealed with a nylon-based lacquer and stored in the dark until scoring.

SUPPLEMENTARY TABLE 1
Protocols for Immunohistochemical Staining

| Target | Antibody | Provider | Dilution |
|---|---|---|---|
| α-catenin | Mouse monoclonal αCAT-7A4 | Zymed | 1:150 |
| Annexin-1/Lipocortin-1 | Mouse monoclonal 29 | Transduction Labs | 1:1000 |
| AP-2α | Rabbit polyclonal K2403 | Santa Cruz | 1:1600 |
| ATF-2 | Rabbit polyclonal C19 | Santa Cruz | 1:250 |
| β-catenin | Mouse monoclonal 14 | Transduction Labs | 1:2500 |
| CD44 | Mouse monoclonal 2C5 | R&D Systems | 1:200 |
| c-Kit | Mouse monoclonal 2E4 | Zymed | 1:50 |
| Connective tissue growth factor | Rabbit polyclonal ab6992 | Abcam | 1:650 |
| E-cadherin | Mouse monoclonal 32 | Transduction Labs | 1:400 |
| Ephrin A1 | Rabbit polyclonal I2203 | Santa Cruz | 1:250 |
| Ephrin receptor Eph A2 | Rabbit polyclonal SC924 | Santa Cruz | 1:200 |
| Fascin | Mouse monoclonal 55K-2 | DAKO | 1:250 |
| Fibronectin (FN1) | Rabbit polyclonal ab299 | Abcam | 1:700 |
| Granulophysin/CD63 | Mouse monoclonal FC-5.01 | Zymed | 1:50 |
| Hairy/Enhancer of Split-related (HEY1) | Rabbit polyclonal | Santa Cruz | 1:200 |
| Human double-minute-2 (HDM2) | Mouse monoclonal 1B10 | Novocastra | 1:100 |
| Integrin-β3/CD61 | Mouse monoclonal SZ21 | Beckman | 1:50 |
| Integrin-linked kinase | Rabbit polyclonal KAP-ST203 | Stressgen | 1:300 |
| Ki-67 | Mouse monoclonal B56 | Transduction Labs | 1:500 |
| L1-CAM | Mouse monoclonal L1-11A | Lab of P. Altevogt | Supernat. |
| MAGE-A1 | Mouse monoclonal MA454 | Zymed | 1:90 |
| Metallothionein (MT-1) | Mouse monoclonal M0639 | DAKO | 1:400 |
| Microphthalmia transcription factor (MlTF) | Mouse monoclonal C5 + D5 | Zymed | Neat |
| Matrix metalloproteinase-1 (MMP-1) | Mouse monoclonal IM35L | Calbiochem | 1:550 |
| Matrix metalloproteinase-3 (MMP-3) | Rabbit polyclonal AB810 | Chemicon | 1:3000 |
| Myelin basic protein | Rabbit polyclonal 18-0038 | Zymed | 1:300 |
| N-cadherin | Mouse monoclonal 3B9 | Zymed | 1:150 |
| Osteonectin/SPARC | Mouse monoclonal AON-5031 | Hematologic Technologies | 1:8000 |
| p120-catenin | Mouse monoclonal 98 | Transduction Labs | 1:400 |
| p16/INK4A | Mouse monoclonal G175-405 | Transduction Labs | 1:500 |
| p21/WAF1/CIP1/CDKN1A | Mouse monoclonal SX118 | Transduction Labs | 1:100 |
| p27/KIP1/CDKN1B | Mouse monoclonal G173-524 | Transduction labs | 1:300 |
| P-cadherin | Mouse monoclonal 56 | Transduction Labs | 1:250 |
| Proliferating cell nuclear antigen (PCNA) | Mouse monoclonal PC10 | Zymed | 1:10,000 |
| Tenascin-C | Rabbit polyclonal SC20932 | Santa Cruz | 1:600 |
| Tissue inhibitor of metalloproteinase-2 (TIMP- | Mouse monoclonal 3A4 | Zymed | 1:75 |
| 2) | | | |
| Tissue inhibitor of metalloproteinase-3 (TIMP- | Mouse monoclonal 136-13H4 | Oncogene Research | 1:10 |
| 3) | | Products | |
| Twist | Rabbit polyclonal H-81 | Santa Cruz | 1:250 |

Automated Quantitative Analysis (AQUA) image acquisition and analysis was performed as previously described (45). Briefly, stained slides were imaged on a modified computer-controlled epifluorescence microscope (Olympus BX-51 with xy-stage and z controller) illuminated by a high-pressure mercury bulb (Photonic Solutions, Missisauga, ON) with a high-resolution monochromatic camera (Cooke Corporation, Romulus, Mich.). Following user optimization of focus, sets of monochromatic, high-resolution (1024×1024, 0.5 μm) images were captured for each histospot for each of the DAPI, Alexa-546 and Cy5 fluorescent channels. Two images were captured for each channel: one in the plane of focus and one 8 μm below it. Compartmentalization of each histospot and quantitation of the target protein signal within each compartment are executed as follows. The Alexa-546 signal representing S100B staining is binary gated to indicate whether a pixel is within the tumor mask (“on”) or not (“off”). Within the region defined by the tumor, the nuclear compartment is defined as the subset of pixels that demonstrated any DAPI staining within the plane of focus. This was required to compensate for the 3-dimensional thickness of the tumor sections which can blur discrimination of the nuclear boundary. The non-nuclear compartment is then defined as all pixels assigned to the tumor mask but are not included within the nuclear compartment. Finally, target antigen expression is automatically determined, blinded to any a priori clinical information, from Cy5 channel the images to obtain relative pixel intensity for the signal emanating from the plane of focus. The final AQUA score for the entire tumor mask or any of its subcellular compartments was calculated as the average AQUA score for each of the individual pixels included in the selected compartment and was reported on a scale of 0 to 255.

Data Management and Statistical Analysis Cores whose tumor mask covered <5% of the total histospot area were dropped from further analysis. For individuals represented by multiple cores on the TMA, AQUA scores were averaged prior to analysis. We normalized AQUA scores between parallel runs of the 2 builds for the Validation Cohort by first calculating a regression equation between the AQUA scores for cell line controls and then adjusting the scores for the Build 1 surgical specimens according to the equation's parameters. The final AQUA score for the Validation Cohort was then calculated as the mean of Build 2 and the adjusted Build 1 AQUA scores. Similarly, to normalize the AQUA scores between the Discovery and Validation Cohorts, a regression equation was calculated for the set of 60 samples spotted on both arrays and the mean values for the Validation Cohort were adjusted according to the regression equation.

For HDM2, Ki-67, MITF, p21 and PCNA, where previous data support nuclear localization of the target in melanoma (47-5), the AQUA score for the nuclear compartment was considered. For AP-2α, ATF-2, p16 and p27, where the ratio of non-nuclear to nuclear expression has previously been shown to have prognostic relevance (46-48), the natural log of the ratio of non-nuclear to nuclear AQUA scores was evaluated in addition to the nuclear (AP-2α, p16, p27) or non-nuclear (ATF2) compartment AQUA score. For the remaining markers, the AQUA scores for the total area under the tumor mask were selected.

The genetic algorithm was executed using the X-tile software suite (49). Briefly, the algorithm randomly selects a set of markers and, for each marker, chooses a random cut-point to binarize the continuous AQUA data, where, by convention, a score of 1 indicates reduced risk and 0 indicates increased risk. Next, for each individual, the binary marker scores are summed and the log-rank statistic for melanoma-specific survival is calculated across all marker sum categories. This initial seed model is then subjected to multiple iterations by either “mutation” (altering the cut-point for an already-included marker) or by “cross-over” (swapping among the set of eligible markers) until the model converges on a maximum likelihood statistic for melanoma-specific survival.

To develop a multi-marker prognostic model from the discovery cohort data, a genetic algorithm using standard methodology (50, 51) within the X-tile software suite (52) with a 33% crossover and 33% mutation rate constrained to create a multi-marker profile that included a minimum of 100/192 eligible individuals with complete data across all selected markers was created. Additional algorithm specifications limited individual marker cut-points to include ≧10% of the available population in each arm and required that each category defined by the marker groupings both contain no fewer than 15% of the available population and, to maintain statistical robustness of the final model, enumerate no fewer than 2 events of interest. We did not constrain the number of parameters to be included in the selected model. Briefly, the algorithm randomly selects a set of markers and, for each marker, chooses a random cut-point to binarize the continuous AQUA data, where, by convention, a score of 1 indicates reduced risk and 0 indicates increased risk. Next, for each individual, the binary marker scores are summed and the log-rank statistic for melanoma-specific survival is calculated across all marker sum categories. This initial seed model is then subjected to multiple iterations by either “mutation” (altering the cut-point for an already-included marker) or by “cross-over” (swapping among the set of eligible markers) until the model converges on a set of markers and their respective cut-points that yield the highest log-rank Chi-square statistic for melanoma-specific survival, typically achieved between 16 and 18 million iterations. Five parallel iterations of the genetic algorithm were executed (see s. Melanoma-specific survival was the end-point for all survival analyses; individuals who died from competing causes were censored at the time of death

Bivariate associations between protein expression in the set of primary tumors with either the metastatic lesions or their associated clinicopathologic criteria were executed using the nonparametric Spearman rank correlation, Mann-Whitney U or Kruskall-Wallis tests. Bivariate associations between the genetic algorithm output and clinicopathologic criteria were evaluated using the chi-square or student's t-tests. Survival curves were calculated using the Kaplan-Meier product-limit method and the log-rank statistic. Univariate and multivariable hazard ratios were calculated using the Cox proportional hazards method, the latter adjusting for known clinicopathologic variables (Breslow thickness, age at diagnosis, gender, stage at diagnosis, microsatellitosis/sentinel lymph node status, tumor site, and receipt of systemic therapy).

Supplemental Results:

Bivariate Relationships Among Markers and with Clinicopathologic Variables

Supplemental FIG. 1 considers all pairwise Spearman Rank correlations (n=136) between markers for the set of primary tumors. Following adjustment for multiple comparisons, 19 positive associations (rₛ≧0.29; p≦0.0003) and one negative association (rₛ≦−0.29) achieved significance. The strongest positive correlations were observed between Ki-67 and p21 (rₛ=0.64) and P-cadherin with each of β-catenin (rₛ0.53) and integrin-linked kinase (r_{ε}=0.53) with the only significant negative correlation observed between Ki-67 and MMP-1 (rₛ=−0.36).

Associations between marker AQUA scores and clinicopathologic characteristics among the primary tumors were performed using non-parametric methods (Supplemental Table 3). Six markers were significantly associated with Breslow thickness; osteonectin, and the ratios for p16/INK4A and p27/KIP1 increased where ATF2, HDM2, and N-cadherin decreased with increasing tumor thickness. None of the associations with the remaining clinicopathologic parameters achieved significance following adjustment for multiple comparisons.

SUPPLEMENTARY TABLE 3
Marker associations with the known clinical prognostic characteristics among the primary tumors

| | | | | | | Tumor- | | |
|---|---|---|---|---|---|---|---|---|
| | | | | | | infiltrating | Chronically | Received |
| | | | | | | lymphocytes | sun | non-surgical |
| | Breslow | Gender | Stage at | Ulceration | Microsatellitosis | (Brisk vs. | exposed site | therapy |
| | (mm) | (M vs. F) | diagnosis | (N vs. Y) | (N vs. Y) | Non-brisk) | (N vs. Y) | (N vs. Y) |
| Target | p-value | p-value | p-value | p-value | p-value | p-value | p-value | p-value |
| α-catenin | p = 0.11 | p = 0.44 | p = 0.10 | p = 0.19 | p = 0.31 | p = 0.07 | p = 0.86 | p = 0.26 |
| Annexin-1/Lipocortin-1 | p = 0.95 | p = 0.21 | p = 0.43 | p = 0.13 | p = 0.03*$ | p = 0.70 | p = 0.08 | p = 0.44 |
| ATF-2 - non-nuclear compartment | p < 0.0001^{#}@ | p = 0.97 | p = 0.25 | p = 0.03@ | p = 0.47 | p = 0.01$ | p = 0.30 | p = 0.19 |
| ATF-2 - in(non-nuclear/nuclear | p = 0.03@ | p = 0.42 | p = 0.55 | p = 0.65 | p-0.45 | p = 0.77 | p = 0.03@ | p = 0.65 |
| compartments) | | | | | | | | |
| β-catenin | p = 0.02@ | p = 0.95 | p = 0.14 | p = 0.13 | p = 0.15 | p = 0.38 | p = 0.82 | p = 0.35 |
| Fibronectin | p = 0.49 | p = 0.24 | p = 0.41 | p = 0.40 | p = 0.22 | p = 0.39 | p = 0.09 | p = 0.10 |
| Hairy/Enhancer of Split-related-1 | p = 0.40 | p = 0.21 | p = 0.69 | p = 0.40 | p = 0.51 | p = 0.91 | p = 0.15 | p = 0.53 |
| Human double-minute-2 - nuclear | p = 0.006*@ | p = 0.92 | p = 0.76 | p = 0.23 | p = 0.30 | p = 0.38 | p = 0.81 | p = 0.56 |
| compartment | | | | | | | | |
| Integrin-linked kinase | p = 0.18 | p = 0.59 | p = 0.86 | p = 0.91 | p = 0.49 | p = 0.55 | p = 0.59 | p = 0.40 |
| Ki-67 - nuclear compartment | p = 0.19 | p = 0.71 | p = 0.03$ | p = 0.35 | p = 0.94 | p = 0.97 | p = 0.79 | p = 0.38 |
| Matrix metalloproteinase-1 | p = 0.49 | p = 0.36 | p = 1.00 | p = 0.75 | p = 0.49 | p = 0.87 | p = 0.29 | p = 0.68 |
| N-cadherin | p = 0.0006^{#}@ | p = 0.66 | p = 0.49 | p = 0.28 | p = 0.71 | p = 0.05$ | p = 0.59 | p = 0.13 |
| Osteonectin/SPARC | p = 0.004*$ | p = 0.55 | p = 0.41 | p = 0.39 | p = 0.89 | p = 0.56 | p = 0.87 | p = 0.27 |
| p16/INK4A - nuclear compartment | p = 0.02 | p = 0.96 | p = 0.94 | p = 0.11 | p = 0.30 | p = 0.25 | p = 0.53 | p = 0.33 |
| p16/INK4A - in(non-nuclear/ | p = 0.007^{#}$ | p = 0.78 | p = 0.92 | p = 0.01$ | p = 0.94 | p = 0.38 | p = 0.51 | p = 0.92 |
| nuclear compartments) | | | | | | | | |
| p21/WAF1/CIP1 - nuclear | p = 0.03$ | p = 0.51 | p = 0.63 | p = 0.12 | p = 0.02$ | p = 0.64 | p = 0.67 | p = 0.16 |
| compartment | | | | | | | | |
| p27/KIP1 - nuclear compartment | p = 0.89 | p = 0.75 | p = 0.69 | p = 0.97 | p = 0.69 | p = 0.16 | p = 0.82 | p = 0.06 |
| p27/KIP1 - in(non-nuclear/nuclear | p = 0.005*$ | p = 0.72 | p = 0.52 | p = 0.02$ | p = 0.29 | p = 0.55 | p = 0.83 | p = 0.73 |
| compartments) | | | | | | | | |
| P-cadherin | p = 0.04@ | p = 0.10 | p = 0.62 | p = 0.15 | p = 0.20 | p = 0.23 | p = 0.62 | p = 0.61 |
| Tenascin-C | p = 0.04@ | p = 0.55 | p = 0.37 | p = 0.27 | p = 0.09 | p = 0.84 | p = 0.31 | p = 0.56 |

*Values significant at p < 0.05 are highlighted in bold text and colored according to the directionality of the association. Positive associations between increasing AQUA score and increased severity of the clinical feature are colored in red and followed by $. Negative associations are colored in blue and followed by @. Associations with female gender are indicated in red and followed by $.
^{#}Significant at the Bonferroni-adjusted p-value of p < 0.0025

Individual Marker Associations with Melanoma-Specific Survival

Methodologically robust multivariable-adjusted individual-protein hazard ratios (52) have been published for 12 markers with our laboratory contributing manuscripts for 6. Among the marginal associations for those 6 not previously reported by our group, only our results for osteonectin/SPARC (no association), nuclear p16/INK4a (improved survival with increased expression), and p27/KIP1 (worsened survival with increased expression) recapitulate previously published results (53-55). Unlike previous reports (53, 54, 56, 57), we did not find a significant association or trend between Ki-67 or Tenascin-C with survival and our significant result for p21 opposed the 1 published result which indicated a trend towards worse survival with increased p21 levels¹⁰. For the remaining 5 markers, these data represent the first report of methodologically robust, multivariable-adjusted survival assessment with our results for Annexin I and Hey-1 being the first instance of IHC data in melanoma.

Descriptive Statistics for the Genetic Algorithm-Based Multi-Marker Prognostic Indicator

One hundred and twenty-nine individuals from the Discovery cohort (67.2%) possessed complete data for all 5 selected markers and were included in the training set. Of these, 20 individuals (15.5%) met all 5 marker conditions, 46 (35.67%) met any 4 of the 5 conditions, 42 (32.6%) met 3 conditions, 19 (14.7%) met 2 conditions and the remaining 2 individuals (1.6%) met 1 condition only. The latter 2 classes were combined in the preliminary algorithm-based groupings. Among the 46 individuals who met 4 of the 5 conditions, we observed an even distribution of the marker not meeting its cut-point with 11 (23.9%) failing the ATF2 ratio, 8 (17.4%) failing β-catenin, 9 (19.6%) failing fibronectin, 10 (21.7%) failing the p16/INK4A ratio and 8 (17.4%) failing p21/WAF1 ruling out any marker-driven selection bias in creating this category.

Bivariate associations between the genetic algorithm-based multi-marker prognostic indicator and the clinicopathologic covariates for the Yale Melanoma Discovery Cohort are reported (Supplemental Table 5). Breslow thickness (2.90 mm±2.10 mm vs. 2.15 mm±1.88 mm; p=0.04) and percentage receiving non-surgical therapy (33.3% vs. 12.1%; p=0.004) were significantly higher among those assigned to the high-risk group. Presence of ulceration (p=0.06) and the lack of a brisk lymphocyte infiltrate (p=0.08) also trended towards significance.

SUPPLEMENTAL TABLE 5
Bivariate associations between the genetic algorithm-derived prognostic
indicator and clinicopathologic correlates of melanoma outcome for the
Yale Melanoma Discovery Cohort.

| | Low-risk group | High-risk group | |
|---|---|---|---|
| Parameter | (n = 66)* | (n = 63) | p-value |
| Breslow thickness (mm) | 2.15 ± 1.88 | 2.90 ± 2.10 | p = 0.04^{†} |
| Age at diagnosis (yrs) | 55.36 ± 15.14 | 57.94 ± 14.67 | p = 0.33 |
| Gender | | | |
| Male | 32 (48.5%) | 31 (49.2%) | p = 0.93 |
| Female | 34 (51.5%) | 32 (48.5%) | |
| Stage at diagnosis | | | |
| Localized | 54 (83.1%) | 52 (83.9%) | p = 0.55 |
| Regional spread | 7 (10.8%) | 4 (6.5%) | |
| Distant metastases | 4 (6.2%) | 6 (9.7%) | |
| Ulceration | | | |
| Absent | 48 (72.7%) | 36 (57.1%) | p = 0.06 |
| Present | 18 (27.3%) | 27 (42.9%) | |
| Tumor-infiltrating | | | |
| lymphocytes | | | |
| Non-brisk | 49 (75.4%) | 55 (87.3%) | p = 0.08 |
| Brisk | 16 (24.6%) | 8 (12.7%) | |
| Histologic subtype | | | |
| Superficial spreading | 46 (69.7%) | 43 (68.3%) | p = 0.98 |
| Nodular | 10 (15.2%) | 12 (19.1%) | |
| Lentigo maligna | 1 (1.5%) | 1 (1.6%) | |
| Acral lentiginous | 4 (6.1%) | 3 (4.8%) | |
| Other | 5 (7.6%) | 4 (6.4%) | |
| Chronically sun-exposed | | | |
| anatomic site | | | |
| No | 30 (45.5%) | 34 (54.8%) | p = 0.29 |
| Yes | 36 (54.6%) | 28 (45.2%) | |
| Received any | | | |
| non-surgical therapy | | | |
| No | 58 (87.9%) | 42 (66.7%) | p = 0.004^{†} |
| Yes | 8 (12.1%) | 21 (33.3%) | |
| Microsatellitosis | | | |
| Absent | 50 (75.8%) | 48 (76.2%) | p = 0.95 |
| Present | 16 (24.2%) | 15 (23.8%) | |

*Numbers may not sum to total due to missing values, percents may not sum to 100% due to rounding.
^{†}Significant at p < 0.05

### REFERENCES

1. Jemal A, Siegel R, Ward P. et al: Cancer statistics, 2008. CA Cancer J Clin 58:71-96, 2008

2. Tsao H, Atkins M B, Sober A J: Management of cutaneous melanoma. N Engl J Med 351:998-1012., 2004

3. Balch C M, Soong S J, Gershenwald J E, at al: Prognostic factors analysis of 17,600 melanoma patients: validation of the American Joint Committee on Cancer melanoma staging system. J Clin Oncol 19:3622-34., 2001

4. Gimotty P A, Elder D E, Fraker D L, et al: Identification of high-risk patients among those diagnosed with thin cutaneous melanomas. J Clin Oncol 25:1129-34., 2007

5, Taylor C: Standardization in immunohistochemistry: the role of antigen retrieval in molecular morphology. Biotech Histochem 81:3-12., 2006

6. Gimotty P A, Van Belle P, Elder D E, et al: Biologic and prognostic significance of dermal Ki67 expression, mitoses, and tumorigenicity in thin invasive cutaneous melanoma. J Clin Oncol 23:8048-56., 2005

7. Gould Rothberg B E, Bracken M B, Rim D L: Tissue biomarkers for prognosis in cutaneous melanoma: a systematic review and meta-analysis. J Natl Cancer Inst in press, 2009

8. Ariyan S, Ariyan C, Farber L R, at al: Reliability of identification of 655 sentinel lymph nodes in 263 consecutive patients with malignant melanoma. J Am Coll Surg 198:924-32., 2004

9. Kononen J, Bubendorf L, Kallioniemi A, et al: Tissue microarrays for high-throughput molecular profiling of tumor specimens. Nat Med 4:844-7., 1998

10. Kreizenbeck G M, Berger A J, Subtil A, et al: Prognostic significance of cadherin-based adhesion molecules in cutaneous malignant melanoma. Cancer Epidemiol Biomarkers Prey 17:949-58, 2008

11. Camp R L, Chung G G, Rimm D L: Automated subcellular localization and quantification of protein expression in tissue microarrays. Nat Med 8:1323-7. Epub 2002 Oct. 21., 2002

12. Mitchell M: An introduction to genetic algorithms. Cambridge, Mass., MIT Press, 1998

13. Ooi C H, Tan P: Genetic algorithms applied to multi-class prediction for the analysis of gene expression data. Bioinformatics 19:37-44, 2003

14. Camp R L, Dolled-Filhart M, Rimm D L: K-tile: a new bio-informatics tool for biomarker assessment and outcome-based cut-point optimization. Clin Cancer Res 10:7252-9., 2004

15. McShane L M, Altman D G, Sauerbrei W, et al: Reporting recommendations for tumor marker prognostic studies (REMARK). J Natl Cancer Inst 97:1180-4, 2005

16. Gimotty P A, Botbyl J, Soong S J, at al: A population-based validation of the American Joint Committee on Cancer melanoma staging system. J Clin Oncol 23:8065-75., 2005

17. Winnepenninckx V, Lazar V, Michiels S, et al: Gene expression profiling of primary cutaneous melanoma and clinical outcome. J Natl Cancer Inst 98:472-82., 2006

18. Viros A, Fridlyand J, Bauer J, et al: Improving melanoma classification by integrating genetic and morphologic features. PLoS Med 5:e120, 2008

19. Bittner M, Meltzer P, Chen Y, et al: Molecular classification of cutaneous malignant melanoma by gene expression profiling. Nature 406:536-40., 2000

20. Alonso S R, Ortiz P, Pollan M, et al: Progression in cutaneous malignant melanoma is associated with distinct expression profiles: a tissue microarray-based study. Am J Pathol 164:193-203., 2004

21. Straume O, Sviland L, Akslen L A: Loss of nuclear p16 protein expression correlates with increased tumor cell proliferation (Ki-67) and poor prognosis in patients with vertical growth phase melanoma. Clin Cancer Res 6:1845-53., 2000

22. Sherr C J, Roberts J M: CDK inhibitors: positive and negative regulators of G1-phase progression. Genes Dev 13:1501-12, 1999

23. Evangelou K, Bramis J, Peros I, et al: Electron microscopy evidence that cytoplasmic localization of the p16(INK4A) “nuclear” cyclin-dependent kinase inhibitor (CKI) in tumor cells is specific and not an artifact. A study in non-small cell lung carcinomas. Biotech Histochem 79:5-10, 2004

24. Keller-Melchior R, Schmidt R, Piepkorn M: Expression of the tumor suppressor gene product p16INK4 in benign and malignant melanocytic lesions. J Invest Dermatol 110:932-8, 1998

25, Sauroja I, Smeds J, Vlaykova T, et al: Analysis of G(1)/S checkpoint regulators in metastatic melanoma. Genes Chromosomes Cancer 28:404-14., 2000

26. Maelandsmo G M, Holm R, Fodstad O, et al: Cyclin kinase inhibitor p21WAF1/CIP1 in malignant melanoma: reduced expression in metastatic lesions. Am J Pathol 149:1813-22, 1996

27. Karjalainen J M, Eskelinen M J, Kellokoski J K, et al: p21(WAF1/CIP1) expression in stage I cutaneous malignant melanoma: its relationship with p53, cell proliferation and survival. Br J Cancer 79:895-902., 1999

28. Bhoumik A, Ronai Z: ATF2: a transcription factor that elicits oncogenic or tumor suppressor activities. Cell Cycle 7:2341-5, 2008

29. Liu H, Deng X, Shyu Y J, at al: Mutual regulation of c-Jun and ATF2 by transcriptional activation and subcellular localization. Embo J 25:1058-69, 2006

30. Bachmann I M, Straume O, Puntervoll H E, et al: Importance of P-cadherin, beta-catenin, and Wnt5a/frizzled for progression of melanocytic tumors and prognosis in cutaneous melanoma. Clin Cancer Res 11:8606-14., 2005

31. Maelandsmo G M, Holm R, Nesland J M, et al: Reduced beta-catenin expression in the cytoplasm of advanced-stage superficial spreading malignant melanoma. Clin Cancer Res 9:3383-8., 2003

32. Banerji A, Das S, Chatterjee A: Culture of human A375 melanoma cells in the presence of fibronectin causes expression of MMP-9 and activation of MMP-2 in culture supernatants. J Environ Pathol Toxicol Oncol 27:135-45, 2008

33. Gaggioli C, Robert G, Bertolotto C, at al: Tumor-derived fibronectin is involved in melanoma cell invasion and regulated by V600E B-Raf signaling pathway. J Invest Dermatol 127:400-10, 2007

34. Jaeger J, Koczan D, Thiesen H J, et al: Gene expression signatures for tumor progression, tumor subtype, and tumor thickness in laser-microdissected melanoma tissues. Clin Cancer Res 13:806-15, 2007

35. Hanahan D, Weinberg R A: The hallmarks of cancer. Cell 100:57-70, 2000

36. Vaisanen A, Kallioinen M, Taskinen P J, et al: Prognostic value of MMP-2 immunoreactive protein (72 kD type IV collagenase) in primary skin melanoma. J Pathol 186:51-8., 1998

37. Vaisanen A H, Kallioinen M, Turpeenniemi-Hujanen T: Comparison of the prognostic value of matrix metalloproteinases 2 and 9 in cutaneous melanoma. Hum Pathol 39:377-385, 2008

38. Rangel J, Nosrati M, Torabian O, et al: Osteopontin as a molecular prognostic marker for melanoma. Cancer 112:144-50, 2008

39. Pacifico M D, Grover R, Richman P I, at al: Development of a tissue array for primary melanoma with long-term follow-up: discovering melanoma cell adhesion molecule as an important prognostic marker. Plast Reconstr Surg 115:367-75, 2005

40. Pearl R A, Pacifico M D, Richman P I, et al: Stratification of patients by melanoma cell adhesion molecule (MCAM) expression on the basis of risk: implications for sentinel lymph node biopsy. J Plast Reconstr Aesthet Surg 61:265-271, 2008

41. Rangel J, Torabian S, Shaikh L, et al: Prognostic significance of nuclear receptor coactivator-3 overexpression in primary cutaneous melanoma. J Clin Oncol 24:4565-9., 2006

42. Paik S, Shak S, Tang G, et al: A multigene assay to predict recurrence of tamoxifen-treated, node-negative breast cancer. N Engl J Med 351:2817-26. Epub 2004 Dec. 10., 2004

43. Ascierto P A, Kirkwood J M: Adjuvant therapy of melanoma with interferon: lessons of the past decade. J Transl Med 6:62, 2008

44. DiVito K A, Charette L A, Rimm D L, et al: Long-term preservation of antigenicity on tissue microarrays. Lab Invest 84:1071-8., 2004

45. Camp R L, Chung G G, Rimm D L: Automated subcellular localization and quantification of protein expression in tissue microarrays. Nat Med 8:1323-7. Epub 2002 Oct. 21., 2002

46. Berger A J, Davis D W, Tellez C, at al: Automated quantitative analysis of activator protein-2alpha subcellular expression in melanoma tissue microarrays correlates with survival prediction. Cancer Res 65:11185-92., 2005

47. Berger A J, Kluger H M, Li N, at al: Subcellular localization of activating transcription factor 2 in melanoma specimens predicts patient survival. Cancer Res 63:8103-7., 2003

48. Denicourt C, Saenz C C, Datnow B, et al: Relocalized p27Kip1 tumor suppressor functions as a cytoplasmic metastatic oncogene in melanoma. Cancer Res 67:9238-43, 2007

49. Camp R L, Dolled-Filhart M, Rimm D L: X-tile: a new bio-informatics tool for biomarker assessment and outcome-based cut-point optimization. Clin Cancer Res 10:7252-9., 2004

50. Mitchell M: An introduction to genetic algorithms Cambridge, Mass., MIT Press, 1998

51. Ooi C H, Tan P: Genetic algorithms applied to multi-class prediction for the analysis of gene expression data. Bioinformatics 19:37-44, 2003

52. McShane L M, Altman D G, Sauerbrei W, et al: Reporting recommendations for tumor marker prognostic studies (REMARK). J Natl Cancer Inst 97:1180-4., 2005

53. Alonso S R, Ortiz P, Pollan M, at al: Progression in cutaneous malignant melanoma is associated with distinct expression profiles: a tissue microarray-based study. Am J Pathol 164:193-203., 2004

54. Straume O, Sviland L, Akslen L A: Loss of nuclear p16 protein expression correlates with increased tumor cell proliferation (Ki-67) and poor prognosis in patients with vertical growth phase melanoma. Clin Cancer Res 6:1845-53., 2000

55. Alonso S R, Tracey L, Ortiz P, et al: A high-throughput study in melanoma identifies epithelial-mesenchymal transition as a major determinant of metastasis. Cancer Res 67:3450-60, 2007

56. Ilmonen S. Jahkola T, Turunen J P, et al: Tenascin-C in primary malignant melanoma of the skin. Histopathology 45:405-11., 2004

57. Niezabitowski A, Czajecki K, Rys J, at al: Prognostic evaluation of cutaneous malignant melanoma: a clinicopathologic and immunohistochemical study. J Surg Oncol 70:150-60., 1999

1. A method for determining the risk that a patient diagnosed with melanoma will develop a recurrence of melanoma comprising:
a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient;
b) determining whether an expression parameter for each marker in the tumor tissue sample is achieved by comparing the level of expression of each marker with a predetermined reference level associated with each marker;
wherein the patient is at a low risk of developing a recurrence of melanoma if four or more of the expression parameters are achieved and wherein the patient is at a high risk of developing a recurrence of melanoma if three or fewer of the expression parameters are achieved.

2. The method of claim 1, wherein the levels of expression of activating transcription factor 2, p21^{WAF1}, p16^{INK4A}, β-catenin, and fibrectin are determined using an automated pathology system.

3. The method of claim 1, wherein the levels of expression of activating transcription factor 2, p21^{WAF1}, p16^{INK4A}, β-catenin, and fibrectin are determined using a quantitative image analysis procedure.

4. The method of claim 1, wherein the melanoma is a stage II cancer.

5. The method of claim 1, wherein the patient diagnosed with melanoma is lymph node negative.

6. The method of claim 1, wherein the compartments of interest are the nuclear compartment and the non-nuclear compartment.

7. A method for determining the risk that a patient diagnosed with melanoma will develop metastatic disease comprising:
a) determining the level of expression for each marker of a panel of markers, wherein the panel comprises activating transcription factor 2, p21^{WAF1}, p16^{INK4A}, β-catenin, and fibronectin and the levels of expression are determined in compartments of interest in cells of interest in a tumor tissue sample from the patient;
b) determining whether an expression parameter for each marker in the tumor tissue sample is achieved by comparing the level of expression of each marker with a predetermined reference level associated with each marker;
wherein the patient is at a low risk of developing metastatic disease if four or more of the expression parameters are achieved and wherein the patient is at a high risk of developing metastatic disease if three or fewer of the expression parameters are achieved.

8. The method of claim 7, wherein the levels of expression of activating transcription factor 2, p21^{WAF1}, p16^{INK4A}, β-catenin, and fibrectin are determined using an automated pathology system.

9. The method of claim 7, wherein the levels of expression of activating transcription factor 2, p21^{WAF1}, p16^{INK4A}, β-catenin, and fibrectin are determined using a quantitative image analysis procedure.

10. The method of claim 7, wherein the melanoma is a stage II cancer.

11. The method of claim 7, wherein the patient diagnosed with melanoma is lymph node negative.

12. The method of claim 7, wherein the compartments of interest are the nuclear compartment and the non-nuclear compartment.

13. A method for determining the risk that a patient diagnosed with melanoma will develop a recurrence of melanoma which comprises:
a) determining the level of expression of activating transcription factor 2 present within a nuclear compartment and a non-nuclear compartment in cells of interest in a tumor tissue sample from the patient;
b) obtaining a ratio of the level of expression of activating transcription factor 2 present within the non-nuclear compartment relative to the level of expression of activating transcription factor 2 present within the nuclear compartment;
c) determining the level of expression of p21^{WAF1} present within the nuclear compartment in the cells of interest in the tumor tissue sample;
d) determining the level of expression of p16^{INK4A} present within the nuclear compartment and the non-nuclear compartment in the cells of interest in the tumor tissue sample;
e) obtaining a ratio of the level of expression of p16^{INK4A} present within the non-nuclear compartment relative to the level of expression of p16^{INK4A} present within the nuclear compartment;
f) determining the level of expression of β-catenin present within the nuclear and non-nuclear compartments combined in the cells of interest in the tumor tissue sample;
g) determining the level of expression of fibronectin present within the nuclear and non-nuclear compartments combined in the cells of interest in the tumor tissue sample;
h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2;
i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1};
j) comparing the ratio obtained in step e) to a predetermined reference ratio associated with p16^{INK4A} wherein the parameter for p16^{INK4A} is achieved if the ratio obtained in step e) is less than or equal to predetermined reference ratio associated with p^{INK4A};
k) comparing the level of expression obtained in step f) to a predetermined reference level associated with β-catenin wherein the parameter for β-catenin is achieved if the level of expression obtained in step f) is greater than the predetermined reference level of expression associated with (-catenin; and
l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin;
wherein the patient is at a low risk of developing a recurrence of melanoma if four or more of the parameters are achieved and wherein the patient is at a high risk of developing a recurrence of melanoma if three or fewer of the parameters are achieved.

14. The method of claim 13, wherein the levels of expression of activating transcription factor 2, p21^{WAF1}, p16^{INK4A}, β-catenin, and fibrectin are determined using an automated pathology system.

15. The method of claim 13, wherein the levels of expression of activating transcription factor 2, p21^{WAF1}, p16^{INK4A}, β-catenin, and fibrectin are determined using a quantitative image analysis procedure.

16. The method of claim 13, wherein the melanoma is a stage II cancer.

17. The method of claim 13, wherein the patient diagnosed with melanoma is lymph node negative.

18. A method for determining the risk that a patient diagnosed with melanoma will develop metastatic disease which comprises:
a) determining the level of expression of activating transcription factor 2 present within a nuclear compartment and a non-nuclear compartment in cells of interest in a tumor tissue sample from the patient;
b) obtaining a ratio of the level of expression of activating transcription factor 2 present within the non-nuclear compartment relative to the level of expression of activating transcription factor 2 present within the nuclear compartment;
c) determining the level of expression of p21^{WAF1} present within the nuclear compartment in the cells of interest in the tumor tissue sample;
d) determining the level of expression of p16^{INK4A} present within the nuclear compartment and the non-nuclear compartment in the cells of interest in the tumor tissue sample;
e) obtaining a ratio of the level of expression of p16^{INK4A} present within the non-nuclear compartment relative to the level of expression of p16^{INK4A} present within the nuclear compartment;
f) determining the level of expression of β-catenin present within the nuclear and non-nuclear compartments combined in the cells of interest in the tumor tissue sample;
g) determining the level of expression of fibronectin present within the nuclear and non-nuclear compartments combined in the cells of interest in the tumor tissue sample;
h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2;
i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1};
j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A};
k) comparing the level of expression obtained in step f) to a predetermined reference level associated with β-catenin wherein the parameter for β-catenin is achieved if the level of expression obtained in step f) is greater than the predetermined reference level of expression associated with β-catenin; and
l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin;
wherein the patient is at a low risk of developing metastatic disease if four or more of the parameters are achieved and wherein the patient is at a high risk of developing metastatic disease if three or fewer of the parameters are achieved.

19-22. (canceled)

23. A method for classifying a patient diagnosed with melanoma as being low risk for a recurrence of melanoma comprising:
a) determining the level of expression of activating transcription factor 2 present within a nuclear compartment and a non-nuclear compartment in cells of interest in a tumor tissue sample from the patient;
b) obtaining a ratio of the level of expression of activating transcription factor 2 present within the non-nuclear compartment relative to the level of expression of activating transcription factor 2 present within the nuclear compartment;
c) determining the level of expression of p21^{WAF1} present within the nuclear compartment in the cells of interest in the tumor tissue sample;
d) determining the level of expression of p16^{INK4A} present within the nuclear compartment and the non-nuclear compartment in the cells of interest in the tumor tissue sample;
e) obtaining a ratio of the level of expression of p16^{INK4A} present within the non-nuclear compartment relative to the level of expression of p16^{INK4A} present within the nuclear compartment;
f) determining the level of expression of β-catenin present within the nuclear and non-nuclear compartments combined in the cells of interest in the tumor tissue sample;
g) determining the level of expression of fibronectin present within the nuclear and non-nuclear compartments combined in the cells of interest in the tumor tissue sample;
h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2;
i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1};
comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A};
k) comparing the level of expression obtained in step f) to a predetermined reference level associated with β-catenin wherein the parameter for β-catenin is achieved if the level of expression obtained in step f) is greater than the predetermined reference level of expression associated with β-catenin; and
l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin;
wherein the patient is at a low risk of developing a recurrence of melanoma if four or more of the parameters are achieved.

24-27. (canceled)

28. A method for classifying a patient diagnosed with melanoma as being high risk for a recurrence of melanoma comprising:
a) determining the level of expression of activating transcription factor 2 present within a nuclear compartment and a non-nuclear compartment in cells of interest in a tumor tissue sample from the patient;
b) obtaining a ratio of the level of expression of activating transcription factor 2 present within the non-nuclear compartment relative to the level of expression of activating transcription factor 2 present within the nuclear compartment;
c) determining the level of expression of p21^{WAF1} present within the nuclear compartment in the cells of interest in the tumor tissue sample;
d) determining the level of expression of p16^{INK4A} present within the nuclear compartment and the non-nuclear compartment in the cells of interest in the tumor tissue sample;
e) obtaining a ratio of the level of expression of p16^{INK4A} present within the non-nuclear compartment relative to the level of expression of p16^{INK4A} present within the nuclear compartment;
f) determining the level of expression of β-catenin present within the nuclear and non-nuclear compartments combined in the cells of interest in the tumor tissue sample;
g) determining the level of expression of fibronectin present within the nuclear and non-nuclear compartments combined in the cells of interest in the tumor tissue sample;
h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2;
i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1};
j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A};
k) comparing the level of expression obtained in step f) to a predetermined reference level associated with β-catenin wherein the parameter for β-catenin is achieved if the level of expression obtained in step f) is greater than the predetermined reference level of expression associated with β-catenin; and
l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin;
wherein the patient is at a high risk of developing a recurrence of melanoma if three or fewer of the parameters are achieved.

29-32. (canceled)

33. A method for determining whether a patient diagnosed with melanoma is likely to benefit from adjuvant therapy comprising:
a) determining the level of expression of activating transcription factor 2 present within the nuclear compartment and the non-nuclear compartment in cells of interest in a tissue sample from the patient;
b) obtaining a ratio of the level of expression of activating transcription factor 2 present within the non-nuclear compartment relative to the level of expression of activating transcription factor 2 present within the nuclear compartment;
c) determining the level of expression of p21^{WAF1} present within the nuclear compartment in the cells of interest in the tissue sample;
d) determining the level of expression of p16^{INK4A} present within the nuclear compartment and the non-nuclear compartment in the cells of interest in the tissue sample;
e) obtaining a ratio of the level of expression of p16^{INK4A} present within the non-nuclear compartment relative to the level of expression of p16^{INK4A} present within the nuclear compartment;
f) determining the level of expression of β-catenin present within the nuclear and non-nuclear compartments combined in the cells of interest in the tissue sample;
g) determining the level of expression of fibronectin present within the nuclear and non-nuclear compartments combined in the cells of interest in the tissue sample;
h) comparing the ratio obtained in step b) to a predetermined reference ratio associated with activating transcription factor 2 wherein the parameter associated with activating transcription factor 2 is achieved if the ratio obtained in step b) is greater than the predetermined reference ratio associated with activating transcription factor 2;
i) comparing the level of expression obtained in step c) to a predetermined reference level associated with p21^{WAF1} wherein the parameter for p21^{WAF1} is achieved if the level of expression obtained in step c) is greater than the predetermined reference level of expression associated with p21^{WAF1};
j) comparing the ratio obtained in step e) to a predetermined reference ratio associated with p16^{INK4A} wherein the parameter for p16^{INK4A} is achieved if the ratio obtained in step e) is less than or equal to the predetermined reference ratio associated with p16^{INK4A};
k) comparing the level of expression obtained in step f) to a predetermined reference level associated with β-catenin wherein the parameter for β-catenin is achieved if the level of expression obtained in step f) is greater than the predetermined reference level of expression associated with β-catenin; and
l) comparing the level of expression obtained in step g) to a predetermined reference level associated with fibrectin wherein the parameter for fibrectin is achieved if the level of expression obtained in step g) is less than or equal to the predetermined reference level of expression associated with fibrectin;
wherein the patient is likely to benefit from adjuvant therapy if three or fewer of the parameters are achieved.

34-37. (canceled)

38. A kit comprising:
a) a first stain specific for activating transcription factor 2;
b) a second stain specific for p21^{WAF1};
c) a third stain specific for p16^{INK4A};
d) a fourth stain specific for β-catenin;
e) a fifth stain specific for fibronectin;
f) a sixth stain specific for a subcellular compartment of a cell; and
g) instructions for using the kit.

39. (canceled)
